(19) United States
(12) Patent Application Publication
(10) Pub. No.: US 2010/0087351 A1
(43) Pub. Date: Apr. 8, 2010

(54) Perfumes for Linear Citrus Release in Rinse-Off Systems

(75) Inventors: Addi Fadel, Jersey City, NJ (US); Michael Gordon Evans, Kent (GB); Grant Mudge, West Redding, CT (US); John Martin Behan, Kent (GB)

Correspondence Address: MORGAN LEWIS & BOCKIUS LLP, 1111 PENNSYLVANIA AVENUE NW, WASHINGTON, DC, 20004, US

(73) Assignee: Quest International Services B.V., Naarden (NL)

(21) Appl. No.: 12/565,868

(22) Filed: Sep. 24, 2009

Related U.S. Application Data
(62) Division of application No. 11/657,698, filed on Jan. 25, 2007.

(30) Foreign Application Priority Data
Jan. 23, 2007 (GB) 0701173.7

(51) Int. Cl. C11D 3/50; A61K 8/34; A61K 8/37

(52) U.S. Cl. 510/107; 512/6; 512/25; 512/26; 510/101

(57) ABSTRACT

Perfume compositions and method of formulating perfume composition are designed for use in wash-off system with a linear citrus perfume release and either any of the following effects: a desired initial release with minimal residual perfume on the targeted system, a long sustained release of fragrance, or a residual deposition of fragrance after use.

### FIELD OF THE INVENTION

This invention relates to the method of the design and engineering of a perfume using odorants' mass transfer properties in order to control the optimization and predicted kinetic progression and/or release of a citrus hedonic profile with time in the presence of high levels of water.

The present invention relates to perfume systems. More particularly, the present inventions relates to the optimization of perfumes used in high water dilution conditions and/or rinse off applications, which will provide a linear continuous citrus hedonic note.

In addition to citrus, this invention provides method to design a predominantly linear citrus hedonic note coupled with a linear secondary nuance of either one of the following odors: fruity, green and floral.

### BACKGROUND OF THE INVENTION

Fragrances are an important part of cosmetic compositions since their primary role is to create an agreeable sensory experience for the consumer, in addition to providing malodor coverage or other more functional roles.

Perfumes are composed of odorants with a wide range of molecular weights, vapor pressures and diffusivities as well as different polarities and chemical functionalities. Using these different properties, an individual skilled in the art could create different hedonic profiles describing the fragrance.

Fragrance materials are generally small molecular weight substances with a vapor pressure that allows their molecules to evaporate, become airborne, and eventually reach the olfactory organ of a living entity. There are a variety of different fragrance materials with different functional groups and molecular weights, 67% both of which affect their vapor pressures, and hence, the ease with which they can be sensed.

Odorants used in perfumery offer a wide array of polarity ranging from the somewhat water miscible to the water immiscible chemical compounds. Perfumery in the various rinse-off applications spanning from cosmetic to industrial and household have different functionalities and must be engineered to fulfill certain needs and objectives. Perfumes' effect and quality during use plays a big role in the consumer's purchase intent as well and the desire of the consumer to purchase the product again.

Fragrances have been designed based upon the selection of odorants with certain properties. For instance, U.S. Pat. No. 6,143,707 directed to automatic dishwashing detergent discloses blooming fragrance compositions by which were chosen based on their clogP and boiling point values. Hydrophobicity is usually gauged by the clogP values of these odorants. The logP value of an odorant is defined as the ratio between its equilibrium concentration in octanol and in water. The logP value of many of the fragrance materials have been reported and are available in databases such as the Pomona92 database, the Daylight Chemical Information Systems, Inc, Irvine, Calif. The logP can also be very conveniently calculated using the fragment approach of Hansch and Leo. See A. Leo, Comprehensive Medicinal Chemistry, Vol 4, C. Hansch et al. p 295, Pergamon press, 1990. These logP values are referred to as clogP values. Odorants thought to result in bloom in water dilutions are thought to have clogP of at least 3.0 and boiling points of less than 26° C. The same rationale for dishwashing liquids with blooming perfumes is also disclosed in U.S. Patent Application Publication No. 2004/0138078. EP Patent No. 0888440B1 relates to a glass cleaning composition containing “blooming perfumes” based on criteria mentioned above. U.S. Pat. No. 6,601,789 discloses toilet bowl cleaning compositions also containing “blooming perfumes” made of odorants chosen based on their clogP values of at least 3.0 and boiling points of less that 260° C. Generally, odorants with delayed bloom are thought to have a clogP of less than 3.0 and boiling point values of less than 250° C.

While the above-mentioned references disclose methods of selecting odorants based upon some of their physical properties, i.e. clogP and boiling point values, they do not encompass and identify all odorants which have superior release properties in heavy water dilutions nor do they provide a quantifying method to define bloom.

Furthermore, descriptors for “blooming odorants” and “delayed blooming odorants” described in the above prior art remain general and do not take in consideration the kinetic aspect of odorants' release in high water dilutions. Predictive quantification of odorants partitioning in headspace based on quantity and various other physico-kinetic aspects are included in the method described herein this invention.

### SUMMARY OF THE INVENTION

A method of formulating a perfume composition for wash-off systems, comprising values of odor detection threshold, an acceleration term (γ) and water release (Ω) values for a group of odorants and engineering the perfume composition in a wash-off system to provide a continuous citrus note upon water dilution.

In addition to citrus, the method enclosed in the herein invention permits the engineering of a linear predominantly citrus perfume in rinse-off coupled with a linear release of secondary, less prominent note of either of the following odor categories: fruity, green, and floral.

The general physical properties of perfume odorants as currently known in the art (e.g., U.S. Pat. No. 6,143,707 U.S. Patent Application Pub. No. 2004/0138078, EP Patent No.0888440 B1 and U.S. Pat. No. 6,601,789) do not provide a complete picture when creating perfumes for rinse-off systems.

Odorants such as ethyl formate, ethyl acetoacetate, ethyl acetate, diethyl malonate, fructone, ethyl propionate, toluic aldehyde, leaf aldehyde, trans-2-hexenal, trans-2-hexenol, cis-3-hexenol, prenyl acetate, ethyl butyrate, hexanal, butyl acetate, 2-phenylpropanal, cis-4-heptenal, cis-3-hexenyl formate, propyl butyrate, amyl acetate, ethyl-2-methylbutyrate, ethyl amyl ketone, hexyl formate, 3-phenyl butanal, cis-3-hexenyl methyl carbonate, methyl phenyl carbinyl acetate, methyl hexyl ether, methyl cyclopentylidene acetate, 1-octen-3-ol, cis-3-hexenyl acetate, amyl vinyl carbinol, 2,4-dimethyl-3-cyclohexen-1-carbaldehyde, ethyl 2-methylpentanoate, 1,3,3-trimethyl-2-oxabicyclo[2.2.2]octane, 3,7-dimethyl-7-methoxyoctan-2-ol etc. are considered by the authors of the herein invention to have superior release properties in heavy water dilutions. Yet, the above mentioned odorants are considered “delayed release” odorants according to the previously mentioned patents, which is counter to both empirical and experimental observations when used in wash-off products.

Prior art mentioned above does not provide ways to quantify bloom or the presence of odorants in headspace in highly diluted water partitions nor do they present a person skilled in the art the ability to predict the kinetic progression of the perfume during rinse-off.

A direct relationship between the quantity of an odorant in a perfume and its ability to be released from the water partition under heavy water dilution is generally observed by perfumers skilled in the art. The opposite can also hold true when using very small amounts of an odorant in a perfume. Above mentioned patents do not account for the change in an odorant's ability to release or bloom due to its concentration or quantity.

A mathematical relationship relating release of odorants from water partitions to their quantity in perfumes as well as their mass transfer properties needs to be established in order to predict their order of elution when exposed to heavy water dilutions.

For example, thiogeraniol (clogP 4.88, boiling point 250° C.) is considered a blooming odorant according to prior art mentioned above. Due to its very low odor detection threshold and overwhelming odor intensity, it is often used as a dilution within a perfume. It can have very delayed water release properties when used in parts per trillion in a perfume although considered a “blooming” material based on its physical properties, according to existing literature and above mentioned patents.

By establishing an approximate correlation mass transfer properties and perceived odorants' hedonic quality and intensity, one can design and further improve water release hedonic perception of perfume materials. The result is the new optimization and applied perfumery for wash off applications.

U.S. Pat. No. 6,858,574 relates odorants release properties in heavy water dilution to a relationship with components of the formulation in which the perfume is delivered, more notably, the surfactant system. The so-called perfume burst index (PBI) is defined by:

$$P B I = \frac{φ - 1.4 / C M C}{K}$$

where φ is water/oil partition

coefficient (an equivalent to clogP mentioned above), K is the volatility constant of perfumes in air (in direct relationship to boiling point values) and CMC is the critical micellization concentration of the surfactant systems (wt/wt). A burst release in water dilutions is thought to happen when there is at least 20% increase of the odorant in headspace. Examples provided by the author are done in dilutions not exceeding 60 and mostly between 0 and 30. Yet, in consumer usage of formulations in wash off conditions, especially in applications such as body wash, conditions, shampoos, and surface cleaners, the conditions far exceed the dilution values used in U.S. Pat. No. 6,858,574 for the calculations. For example, a typical usage of water during a shower exceeds 25 gallons of water and can reach 50 gallons of water when considering a typical household shower pressure dispensing 5-10 gallons a minute (See

http://www.engr.uga.edu/service/extension/publications/c819-1.html).

Values for water dilutions in a typical household, cosmetic, industrial wash-off application therefore far exceeds the dilution values used in U.S. Pat. No. 6,858,574. One can therefore argue that under these extreme dilution conditions of a typical wash-off application (1/100 and above), the release partitions become essentially water, water-air and air, with surfactants' contributions very minimal, almost non-existent.

In the present invention, mass transfer properties of odorants in water as well as their odor detection threshold values and hedonic descriptors are used to design fragrances optimized for rinse-off.

Descriptors of fragrance ingredients are designated under two categories (“Descriptor 1” and “Descriptor 2”) independently by a panel of in-house expert perfumers. Descriptor 1 is used to describe the overall dominating hedonic perception whilst descriptor 2 is mostly for nuances of the odorant. By definition, citrus or fruity, green, and floral odorants will be defined as such, preferably based on either one or the other of the descriptors, and more preferably “Descriptor 1”.

Specific physico-chemical properties of odorants are utilized in methods described in this invention to control and engineer linear citrus hedonic perfumes during use or alternatively linear citrus-fruity, citrus-floral and citrus-green.

According to the present invention, a perfume composition is optimized for various cosmetic, household and industrial applications in water systems and/or in presence of water based on specific physico-kinetic properties. In addition, methods are included to estimate odorants' hedonic contribution (odor being defined by the said odorants' odor descriptors) out of a total odor value within specifically designated water release groupings.

The perfumes of this invention are also designed to potentially give the consumer the perception of sustained and more prolonged release of a citrus perfume or citrus-fruity, citrus-green and citrus-floral during wash-off. Methods to create such superior sustained citrus release in high water dilutions are used for perfumes used in cosmetic and household applications.

The perfumes created using methods described herein this invention also have the ability provide a linear release of a citrus fragrance or a predominantly citrus fragrance with a secondary fruity or floral or green odor also in a linear manner without substantial residual perfume left behind on a surface upon the completion of the wash-off experience. This desired effect will target certain applications such surface cleaners and dish washing liquids.

Perfumes engineered according to methods described in this invention can also provide the person skilled in the art with a method to create a sustained release of a perfume with a constant perceived intense citrus background upon heavy dilution with linear nuances of fruit, green and floral. Such perfumes are intended for household and cosmetic applications such as shampoo, conditioners, body wash and soaps.

Finally, other important categories of cosmetic, household and industrial rinse-off products must result in a substantial deposition of perfumes upon rinse-off. Methods to create such perfumes with an additional intense background of a citrus perfume throughout the rinse-of experience are shown herein. These perfumes can also be designed as mentioned in the above cases to include linear nuances of floral, green or fruity perfumes against a strong citrus background during rinse-off.

This invention deals primarily with the method to optimize a citrus fragrance diffusion and behavior in high water dilutions based on calculated mass transfer and transport properties of odorants in water, water vapor and air partitions according to methods described herein.

The object of this patent is to improve a citrus fragrance released perception during delivery or release in presence of large volumes of water.

In water-based systems, choosing fragrance molecules based on specific mass-transfer values for release out of a matrix optimizes the perfume's intensity and perceived hedonic quality. These values are calculated according to these odorants' physico-chemical properties based on principles of mass transfer as well as odorants calculated odor contributions within defined water release groups.

Water release value (Ω) is defined by the authors as being the product of quantity of an odorant in a perfume totaling 100 parts used arbitrarily at 1% in rinse-off application with the odorant's flux (φ) and its estimated pseudo-acceleration value (γ) out of the water-air partitions.

These Ω values are used to separate the fragrance into water release groups, therefore predicting the kinetic release of odorants out the water, water/air into the air partitions.

Within these defined water-release groups, odorants are then further described based on their experimentally determined odor detection thresholds (ODT) to further characterize the odor impact or olfactive intensity of a citrus and other olfactive types within the herein-described released group of odorants.

Based on the application considered, the perfume considered will be optimized using odorants' mass transfer and physico kinetic properties as well as their odor intensity and description. “Water release groups” for water partitions are defined in more details in the invention and are engineered specifically to result in fragrances with an impactful citrus background during the entire rinse-off experience.

Perfumes designed for surface cleaners and dishwashing detergents are composed of at least 20%, preferably at least 30% of total perfume odorants with characteristic flash water release values: γ values more than 900 and in addition, no more than 30%, more preferably no more than 15% of the composing odorants must have γ values below 100.

Perfumes engineered for shampoos, conditioners, body wash etc. will on the other hand be optimized using primarily sustained release odorants based on the optimal residence time in headspace. Fragrances constructed with at least 30% and preferably at least 40% of odorants with acceleration values for sustained release: γ values between 900 and 100.

More residual fragrances for wash-off applications such as laundry can be engineered based on a majority of fragrance at least 30%, preferably 40% of odorants, more preferably 50%, referred to by the authors as “deposition odorants,” based on their mass transfer properties: γ values lower than 100.

According to the present invention, all perfumes engineered for intended functionalities described above will provide a continuous citrus fragrance during rinse-off or in the presence of large quantities of water. Green, fruity and floral nuances may also be built in the linear release of the perfume out of the rinse-off partitions, essentially creating what the inventors refer to as linear “citrus-green”, “citrus-fruity”, and “citrus-floral” blooming perfumes.

A continuous, sustained citrus hedonic background during rinse off can be achieved designing at least three, preferably four different release groups as described in this invention with at least 30%, preferably at least 40% of their total odor contributed by one or a group of citrus odorants.

In addition, linear release of olfactive floral, green and fruity nuances may be added to a dominating citrus background during rinse off. To achieve secondary linearity of either floral, green or fruity nuances, one or a group of the corresponding floral, green or fruity odorants must contribute to at least 20%, of at least three, preferably four different water release groups as described in the invention herein.

### Water Release Value, Ω

Water based formulations are usually oil in water or water in oil emulsions with a varied concentration of water. By emulsifying these partitions, fragrances are dispersed and solubilized. Upon heavy water dilutions typical for the average household, industrial and cosmetic rinse-off in-use, odorants making up perfumes need to diffuse through what is considered to be mostly water, a vapor phase above the liquid phase and finally the air phase.

To increase the water release impact of these fragrances in these systems, properties of odorants based on their mass transfer characteristics were used. These odorants' release properties in water (Ω_{1,2}) will determine the order of elution of these odorants in the partitions considered: water, water-air and air

Ω=n·φ·γ [1]

φ=Flux of odorant in a system considering the partitions: water, water-air and air, expressed in

$$\frac{mg}{{cm}^{2} \times sec}$$

and
γ=Pseudo-acceleration factor of odorant in water, water-air and air expressed in

$$\frac{cm}{{sec}^{2}} ,$$

n is the parts quantity of an odorant in a total 100 parts of a perfume used arbitrarily at 1% in a formulation.

This value of water release is indicative of the kinetic order of elution of the odorants involved in the composition of the perfume diluted in water. As discussed later in this document, it is intimately linked to various thermodynamic and calculated mass transfer properties obtained by the authors but also based on quantity of the odorant considered within the entire formula.

Below is the description of the terms used to derive equation [1]

### Flux (φ₁₂)

Flux of an odorant in partitions water, water-air and air, (φ) is defined as the ratio of the quantity of odorant being transferred in the media considered divided by the time and area of the contained medium. Flux values can also be defined in relation to a concentration gradient of the odorant throughout a partition according to:

$${φ}_{12} = - {D}_{12} ( \frac{d ( {c}_{1} )}{d z} ) \qquad [ 2 ]$$

D₁₂ is the diffusion constant of odorant (1) in partition (2) and dC₁/dZ is the concentration gradient of odorant (1) throughout the partition.

D₁₂ is calculated using the “Slattery Kinetic Theory” with non-polar odorants using odorants' critical parameters, unsteady state evaporation and measurement of binary diffusion coefficient. (Chem. Eng. Sci. 52, 1511-1515). The concentration gradients of the odorants composing the perfumes throughout the partitions considered (water, water-air and air) are calculated by solving for the dimensionless velocity value determined using the Arnold equation. (See Arnold, J. H. Studies in Diffusion: III. Unsteady State Vaporization and Absorption. Trans. Am. Inst. Chem Eng., 40, 361-378.). Some flux values for a variety of odorants out of a water partition are listed in the Table 1 below.

TABLE 1
Examples of flux values for some perfume odorants.

| Odorant | φ (mg/cm² · sec) |
|---|---|
| Ethyl 2-methylbutyrate | 0.004361536 |
| d-1-Methyl-4-isopropenyl-1-cyclohexene | 0.001571820 |
| 2,2-Dimethyl-3-(p-ethylphenyl)propanal | 0.000006157 |
| 4-Methyl-3-decen-5-ol | 0.000004491 |
| 5-Hexyldihydro-2(3H)-furanone | 0.000005070 |
| 1-(5,5-Dimethyl-1-cyclohexen-1-yl)-pent-4-en-1-one | 0.000005501 |
| 6,6-Dimethyl-2-methylenebicyclo-(3.1.1)-heptane | 0.001912106 |
| 6-sec-Butylquinoline | 0.000006754 |
| Octahydro-4,7-methano-1H-indene-5-yl acetate | 0.000009115 |
| Ethyl 2,3-epoxy-3-methyl-3-phenylpropionate | 0.000010182 |
| 2(6)-methyl-8-(1-methylethyl)-bicyclo[2.2.2]octe-5-en-2(3)-yl-1,3-dioxolane | 0.000003792 |
| Isopropyl-methyl-2-butyrate; | 0.002632239 |
| Tricyclo-decenyl propionate | 0.000003150 |
| 2,6,10-Trimethyl-9-undecenal | 0.000001843 |
| Methyl-2-hexyl-3-oxocyclopetanedecarboxylate | 0.000000204 |
| 2-Phenylethyl phenylacetate | 0.000000080 |
| 3,7-Dimethyl-1,6-octadien-3-yl 3-phenyl-2-propenoate | 0.000000039 |
| Ethyl octyne carbonate | 0.000007735 |
| 3,7-Dimethyl-2,6-octadien-1-thiol | 0.000046576 |
| (1R-(1a,4b,4aa,6b,8aa))-Octahydro-4,8a,9,9-tetramethyl-1,6-methano-1(2H)-naphtol | 0.000001119 |

### Pseudo-Acceleration, γ₁₂

In the analysis of the volatility of odorants, several variables are found to be important. First, the vapor pressure of the odorant is an important measure of its volatility. The product of the odorant's activity coefficient γ in the partition, its mole fraction X and its pure vapor pressure value Pᵥ, gives the odorant's relative vapor pressure. A second important factor for volatility is the diffusivity D₁₂ of the odorant in the considered media: water, vapor phase and subsequently air.

Other important variables to consider are the molecular weight M_{w}, of the odorant and its density in the partition pi and in the solvent vapor state ρᵥ. The final variable to consider is an energy parameter in the partition state. The energy difference ε₁₂=ε₁₂₍ₚₒₗₐᵣ₎−ε₁₂ₒ₍ₙₒₙ₋ₚₒₗₐᵣ₎ is proportional to the partition coefficient of an odorant in a polar solvent such as water, and a water immiscible solvent such as octanol, benzene and paraffin liquid. The energy ε₁₂ is called the partition energy and can be correlated to the clogP value of odorants. By definition: clogP proportional to

$$\frac{( {ɛ}_{12 ( water )} - {ɛ}_{12 ( octanol )} )}{RT} ;$$

R=1.987 cal/(mole-°K); T=temperature (Kelvin).

The five variables D₁₂, Pᵥ, Mw, ρᵥ, and ε₁₂ and the three dimensional variables indicate that there can be 5−3=2 dimensional variables which describe Newton's law. The easiest separation is to break the acceleration vector into 2 dimensional quantities: a frequency or first order rate constant (1/time) and a velocity (distance/time) term.

The velocity group can be formed from the vapor pressure and density. Since pressure has units of (mass*distance)/(distance²*time²), and density has units of mass/distance³, the ratio of the two has units of velocity squared. The square root gives the desired velocity. This velocity group is therefore defined as:

$$Velocity = {( \frac{γ * X * {P}_{v}}{{ρ}_{v}} )}^{\frac{1}{2}} \\ ( Units \text{:} length \text{/} time ) \qquad [ 3 ]$$

The first order rate constant can be formed from the variables Mw, D₁₂ and ε₁₂. Since the partition energy ε₁₂ has dimensions of calories per mole (mass.length²/mole.time²) and the diffusivity coefficient D₁₂ has a dimension of distance² per time, the ratio yields exactly a molecular weight unit per time t. The energy can be made dimensionless by dividing by the gas constant k and temperature T. The remaining variable D₁₂ can be made to a frequency by dividing by a cross sectional area L². A molecular area calculated from the liquid molar volume could represent this area. The frequency term or first order rate constant is therefore defined as:

$$Frequency = \frac{{ɛ}_{12}}{MW * {D}_{12}} \\ ( Units \text{:} 1 / time ) \qquad [ 4 ]$$

Some γ values for a variety of odorants are listed below in Table 2.

TABLE 2
Calculated pseudo-acceleration values for some perfume odorants

| Odorant | γ (cm/sec²) |
|---|---|
| Ethyl 2-methylbutyrate | 12827.56 |
| d-1-Methyl-4-isopropenyl-1-cyclohexene | 8200.76 |
| 2,2-Dimethyl-3-(p-ethylphenyl)propanal | 121.17 |
| 4-Methyl-3-decen-5-ol | 116.38 |
| 5-Hexyldihydro-2(3H)-furanone | 115.36 |
| 1-(5,5-Dimethyl-1-cyclohexen-1-yl)-pent-4-en-1-one | 109.12 |
| 6,6-Dimethyl-2-methylenebicyclo-(3.1.1)-heptane | 9007.51 |
| 6-sec-Butylquinoline | 135.34 |
| Octahydro-4,7-methano-1H-indene-5-yl acetate | 144.06 |
| Ethyl 2,3-epoxy-3-methyl-3-phenylpropionate | 147.67 |
| 2(6)-methyl-8-(1-methylethyl)-bicyclo[2.2.2]octe-5-en-2(3)-yl-1,3-dioxolane | 57.74 |
| Isopropyl-methyl-2-butyrate; | 8722.05 |
| Tricyclo-decenyl propionate | 60.58 |
| 2,6,10-Trimethyl-9-undecenal | 43.58 |
| Methyl-2-hexyl-3-oxocyclopetanedecarboxylate | 6.71 |
| 2-Phenylethyl phenylacetate | 2.29 |
| 3,7-Dimethyl-1,6-octadien-3-yl 3-phenyl-2-propenoate | 0.71 |
| Ethyl octyne carbonate | 156.29 |
| 3,7-Dimethyl-2,6-octadien-1-thiol | 659.09 |
| (1R-(1a,4b,4aa,6b,8aa))-Octahydro-4,8a,9,9-tetramethyl-1,6-methano-1(2H)-naphtol | 25.57 |

Pseudo acceleration values are also closely linked to the ability of an odorant to travel through headspace once it is airborne in addition to its ability to migrate through the water and water-air partitions. This value is predictive of what the authors consider “flash release”, “sustained release” and “deposition” of odorants in heavy water dilutions.

“Flash release” is defined as fast migration through water and subsequent very low residence time in headspace, resulting in a short hedonic experience of initial release and very minimal deposition on a treated surface. “Sustained release” is characterized by good water release properties along with a longer residence time in the water vapor and subsequently, the air phase. “Deposition” or also “delayed-release” is a term used to categorize odorants with very poor water/air release properties and consequently remain available for superior deposition on the surfaces treated.

Flash release odorants are considered by the authors to have acceleration, γ values above 900 cm/sec², sustained release odorants are thought to have γ values between 900 and 100 and finally deposition odorants have acceleration values of less than 100.

As an illustration, some odorants with characteristic acceleration values for all three release categories defined by the authors are shown below. Water release properties are observed in 1 to 100 water dilution of a typical formulation containing these odorants as shown in the following procedure. The odorants chosen for this illustrative example are as follow in Table 3.

TABLE 3
Release properties and predicted residence
time for some perfume odorants.

| | | Γ (acceleration water/air) | |
|---|---|---|---|
| Flash Release | ethyl formate | 46183.23 | cm/sec² |
| | ethyl-2-methyl butyrate | 12827.56 | |
| | melonal | 2655.52 | |
| | cyclacet | 1687.87 | |
| Sustained Release | linalool | 644.41 | |
| | aldehyde c-11 moa | 401.44 | |
| | alpha ionone | 283.60 | |
| | lilial | 104.63 | |
| Deposition Odorants | cyclamen aldehyde | 99.64 | |
| | jasmolactone | 76.30 | |
| | hexyl cinnamic aldehyde | 21.01 | |
| | acetal cd | 0.08 | |

The partition release value Ω is defined as the product of the pseudo acceleration γ and the flux value Φ and the quantity of odorant in a total 100 parts of the perfume diluted in water. The expression of water release out of the water, water-air and air partitions can then be physically equated to a value of

$$( \frac{force}{area} ) \frac{1}{sec}$$

or in other words, units of pressure per time out partition. It is important to establish that water release values are a way to predict the kinetic release profile of a perfume out the partitions considered into headspace when subject to extreme aqueous dilutions. This predictive value for elution time allows a person skilled in the art to establish groupings of odorants as they kinetically elute from the water dilutions. Keys or hedonic profile can be constructed, achieving better engineering control of their creative process. By designing these groupings of odorants and their order of elution, a perfumer can construct optimized perfumes for water release systems, since most of these odorants will behave differently in aqueous dilutions as compared to emulsions with various surfactant proportions.

Water release values, Ω for the corresponding odorants is a kinetic expression of water release. Once in headspace, acceleration values as well as odor detection thresholds (discussed in more details further) will dictate the intensity and odor contribution as well as residence time of each odorant in the water vapor and air.

An empirical relationship using real time headspace analysis was established by the authors between elution times of odorants and Ω values. This empirical relationship is shown in Table 5.

TABLE 5
Water Release Groups Definitions.

| | Water Release Values |
|---|---|
| Water Release Group 1 | Ω ≧ 10 |
| Water Release Group 2 | 10 > Ω ≧ 0.07 |
| Water Release Group 3 | 0.07 > Ω ≧ 0.007 |
| Water Release Group 4 | 0.007 > Ω ≧ 0.0005 |
| Water Release Group 5 | 0.0005 > Ω ≧ 0.00003 |
| Water Release Group 6 | 0.00003 > Ω |

Examples of odorants having an acceleration value greater than 900 include:
ethyl formate
ethyl acetate
ethyl propionate
ethyl 2-methylpropanoate
methyl hexyl ether
2,6,6-Trimethylbicyclo-(3,1,1)-2-heptene
butyl butyrate
ethyl isovalerate
ethyl butyrate
ethyl 2-methylbutyrate
butyl acetate
hexanal
isopropyl-methyl-2-butyrate;
β-methyl butyl acetate
6,6-dimethyl-2-methyleneorpinene
pentyl acetate
propyl butyrate
7-methyl-3-methylene-1,6-octadiene
(R)-(+)-p-Mentha-1,8-diene
2,6-Dimethyl-2-heptanol
2-ethenyl-2,6,6-trimethyltetrahydropyran
E-2-hexenal
4-isopropyl-1-methyl-1,5-cyclohexadiene
cis-4-heptenal;
methyl phenyl ether
1-methyl-4-isopropyl-1,4-cyclohexadiene
ethyl-2-methylbentanoate
3-methyl-2-butenyl acetate
hexyl formate
1-methyl-4-isopropylidene-1-cyclohexene
1,3,3-trimethyl-2-oxabicyclo[2.2.2]octane
2,3-butanedione
3,7-dimethyl-1,3,6-octatrene
ethyl hexanoate
cis-3-hexenyl formate
6-methyl-5-hepten-2-one
3-octanone
trans-2-hexenyl acetate
2,2-Dimethyl-3-(3-methyl-2,4-pentadienyl)-oxirane
2-(2′-methyl-1′-propenyl)-4-methyltetrahydropyran
octanal
hexyl acetate
methyl-2,2-dimethyl-6-methylene-1-cyclohexanecarboxylate
phenylethyl methyl ether
methyl phenyl carbinyl acetate
3,3-dimethyl-8,9-dinorbornan-2-one
isobutyl cis-2-methyl-2-butenoate
cis-4-(isopropyl)-cyclohexane methanol
isoamyl butyrate
2,6-dimethyl-2hepten-7-al
pentyl butyrate
tricyclo decenyl acetate
5-methyl-2-(2-methylpropyl)-cis-3-Propylbicyclo(2.2.1)hept-5-ene-2-carbaldehyde
Methyl trans-1,4-dimethylcyclohexanecarboxylate
1,3-Dimethylbutyl 2-butenoate
4-(1-Methoxy-1-methylethyl)-1methylcyclohexene
2-Methyl-1,5-dioxaspiro[5,5]undecane
3,6-Dihydro-4methyl-2-(2-methylopropen-1-yl)-2H-pyran;
2-Propenyl hexanoate
cis-3-hexenyl isobutyrate
ethyl heptanoate
2,4dimethyl-3-cyclohexen-1-carbaldehyde
cis-3hexenyl methyl carbonate;
1-Ethyl-3-methoxytricyclo[2.2.1 02,6]heptane
1-(3,3-Dimethylcyclohexyl)ethan-1-one
nonanal
trans-2-hexenol
dl-1,7,7-Trimethylbicyclo[2.2.1]heptan-2-one
1,3Dimethylbut-3-enyl isobutyrate
cis-3-hexenol
3,7-dimethyl-7-methoxyocian-2-ol
Methyl cyclopentylidene acetate
benzaldehyde
Aldehyde C-8 dimethyl acetal
3,7-Dimethyl-1,6-octadien-3-yl formate
3,7-Dimethyloctanal
2,6-dimethyl-2-heptanol
4,5,6,7-Tetrahydro-3,6-dimethylbenzofuran
1,3,5-Undecatriene
2,5-dimethyl-2-octen-6-one
cis-3-hexenyl acetate
butyl 2-methyl pentanoate
3,7-Dimethyl-6-octenal
dimethyloctenone;
2,4-Dimethyltetrahydro benzaldehyde
cis-3-hexanyl propionate
2-isopropyl-5-methylcyclohexanone (isomer unspecified;
2-(1-Ethylpentyl)-1,3-dioxolane
3-octanol
2-phenylpropanal
3,5,5-trimethyl hexanal
1,3-undecadein-5-yne
1p-menthene-8-thiol;
1Phenyl-4-methyl-3-oxapentane
3,7-Dimethyl-3,6-octadienal
3-Octenol
E-4-Decenal
cis-4-decenal
phenylacetaldehyde
2-(1-methylpropyl) cyclohexanone
2-Butyl-4,4,6-trimethyl-1,3-dioxane
cyclohexyl ethyl acetate
1-octen-3-ol
tricyclodecenyl propionate
6-Butyl-2,4-dimethyldihydropyrene
2,6-nonadienal
3-phenyl butanal
3,7-dimethyl-2,6-octadiene-1-nitrile
Z-6-nonenal
   Examples of odorants having an acceleration value less than 100 include:
2-isobutyl-4-methyltetrahydro-2H-pyran-4-ol
o-Amino methylbenzoate
1-(2,6,6-Trimethyl-2-cyclohexene-1-yl)-1,6-heptadien-3-one
3,7-Dimethyl-6-octenyl 3-methylbutanoate
4-Methoxybenzaldehyde diethyl acetal
[2-(Cyclohexyloxy)ethyl]benzene
AGARBOIS
2-Methoxy-4-(2-propenyl)phenol
2(6)-methyl-8-(1-methylethyl)bicyclo[2.2.2]ocle-5-en-2(3)-yl-1,3-dioxoiane
2-Methyl-4-(2,2,3-trimethyl-3-cyclopenten-1-yl)-2-buten-1-ol
3-Phenylpropyl alcohol
2-(Phenylmethylene)heptanal
Ethyl (2E,4Z)-decadienoate
7-Methyl-2H-benzo-1,5-dioxepin-3(4H)-one
Ethyl 2-hexylacetoacetate
4,4a,5,9b-Tetrahydroindeno[1,2-d]-1,3-dioxine
3-Methyl-5-phenylpentanenitrile
3,4-Dihydro-2H-1-benzopyran-2-one
2-Phenoxyethyl isobutyrate-Dodecanenitrile
2-(3-Phenylpropyl)pyridine
2,6,19-trimethyl-5,9-undecadienal
p-isobutyl-a-methyl hydrocinnamaldehyde
trans-3,7-Dimethyl-2,8-octadien-1yl 3-methylbutanoate
8-β-H-Cedran-8-ol acetate
VETHYMINE
Tricyclo(5.2.1.02.6)dec-3-en-9-yl isobutyrate
Trimethyl-13-oxabicyclo[10.1.0]trideca-4,8-diene
3,7-Dimethyl-7-hydroxyoctanan
2-Benzyl-4,4,6-trimethyl-1,3-dioxene
amberketal;
2,6,10-Trimethyl-9-undecenal
γ-undecalacione
10-undecen-1-ol
1,2-Benzopyrone
4-(p-Methoxyphenyl)-2-butanone
3-Butyltetrahydro-5-methyl-2H-pyran-4-ylacetate
3(Or 4)-(4-methylpenten-3-yl)cyclohex-3-ene-1-methyl acetate
6,10-dimethyl-9-undecen-2-one
carbonic acid:4-cyclootene-1-ylmethyl ester;
2-(2-Methylphenyl)ethanol
a,a-Dimethylphenethyl butyrate
4-Hydroxy-3-methoxy-1-propenylbenzene
1,5,5,9-Tetramethyl-13-oxatricyclo(8,3,0,0(4,9))iridecanel
2-Methyl-4-(2,2,3-trimethyl-3-cyclopentenyl)butanol
2-isobutoxynaphtalene
3,7,11-Trimethyl-2,6,10-dodecatrien-1-ol
Methoxy dicyclopentadiene carboxaldehyde
1,1′-Bicyclopentyl]-2-yl2-buteneate; 2-Cyclopentylcyclopentyl cretonate;
methyl-2-naphtyl ketone
1,2,3,4,4a,5,6,7-Octahydro-2,5,5-trimethyl-2-naphthol
2H-Pyran-2-one, tetrahydro-8-(3-pentenyl)
FRESCILE
Dihydro-5-octylfuran-2(3H)-one
1,2,3,4,4a,7,8,8a-Octahydro-2,4a5,8a-tetramethyl-1-naphthyl formate
FRUTONILE
magnolan;
3-Methyl-5-phenylbentanol
(E) and (Z) 6,10-Dimethylundeca-5,9-den-2-yl acetate
alcohol C-12, dodecanol
5,6-Dimethyl-β-isopropenylbicyclo(4.4.0)dec-1-en-3-one
2-methyl-5-phenlpentanol
3-methyl-5-phenlpentanol
2-Methoxy-4-propenylphenyl acetate
1-(1,2,3,4,5,6,7,8-Octahyro-2,3,8,8-tetramethyl-2-naphthalenyl)ethanone
Tricyclo[6.3.1.02.5]dodecan-1-ol, 4,4,8-trimethyl-, acetate, [1R-(1a, 2a,5b,8b)]-;
PIVACYCLENE
Ethyl a,b-epoxy-b-phenylpropionate
3-(4-ethyl phenyl)-2,2-dimethylproapanenitrile
(1R-(1a,4b,4aa,6b,8aa))-Octahydro-4,8a,9,9-tetramethyl-1,6-methano-1(2H)-naphthol
2-methyl-3-(3,4-methylenedioxyphenyl)propanal
3-Methylbutyl o-hydroxybenzoate
2-Ethyl-4-(2,2,3-trimethyl-3-cyclopenten-1-yl)-2-buten-1-ol
1,3-Benzodioxole-5-carboxaldehyde;
benzyl alcohol
1-Phenyl-3-methyl-3-pentanol;
2-Ethyl-2-prenyl-3-hexenol
4-Acetyl-6-t-butyl-1,1-dimethylindan;
α-hexylcinnamic aldehyde;
2-Oxo-1,2-benzopyran,
3aR-(3aa,5ab,9aa,9bb)Dodecahydro-3a,6,6,9a-tetramethylhaphtho(2,1-b)furan,
hydroxycitronellal dimethyl acetal
2-Methyl-4-phenylpentanol;
3,7,11-Trimethyldodeca-1,6,10-trien-3-ol mixed isomers
a,b,2,2,3-Pentamethylcyclopent-3ene-1-butanol
3,12-tridecadien-nitrile
3a,4,5,6,7,7a-Hexahydro-2,6(3,6)-dimethyl-4,7-methano-1H-inden-5-ol
3-Phenyl-2-propen-1-ol
4-(2,6,6-Trimethylcyclohexyl)-3-methylbutan-2-ol;
4-(3,4-Methylenedioxyphenyl)-2-butanone;
3,4-dimethoxybenzaldehyde,
SINODOR
3-Methyl-5-(2,2,3-trimethyl-3-cyclopenien-1-yl)pent-4-en-2-ol
Ethoxymethoxy)cyclodecane;
2-ethoxy-4-methoxymethylphenol,
2-[2-(4-Methylcyclohex-3-en-1yl)propyl]cyclopentanone;
4-(4,8-Dimethylnona-3,7-dienyl)pyridine
(E,E,E)-2,6,10-Trimethyldodeca-2,6,9,11-tetraen-1-al
DUPICAL
Methyl 3-phenylpropenoate
7-Methyl-2H-benzo-1,5-dioxepin-3(4H)-one
amber core;
3-(2-bornyloxy)-2methyl-1-propanol (exo)
3Phenyl-2-propen-1-yl 3-methylbutanoate
trans-2,4-Dimethyl-2-(5,6,7,8-tetrahydro-5,5,8,8-tetramethyl-2-naphthalenyl)-1,3-dioxolan
a-Cyclohexylidene benzeneacetonitrile,
3-(Hydroxymethyl)nonan-2-one;
Benzoic acid, 2-hydroxy-, 3-methyl-2-butenyl ester
cedryl methyl ketone
cis-4-Cyclopentadecanone;
6-Ethylidineociahydro-5,8-methano-2H-1-benzopyran-2-one;
5-cyclohexadecen-1-one;
cyclopentadecanone;
MEVANTRAAL
3,3-Dimethyl-5-(2,2,3-trimethyl-3-cyclopenten-1-yl)-4-penten-2-ol
methyl dihydrojasmonate
cyclopentadecanoide
1,3-Dioxane, 2-(2,4-dimethyl-3-cyclohexene-1-yl)-5-methyl-5-(1-methylpropyl)-3,7-dimethyl-1,6-octadien-3-yl benzoate.
Methyl(2-pent-2-enyl-3-oxo-1-cyclopentyl) acetate
2-tert-butylcyclohexyl carbonate;
4-(4-hydroxyphenyl)-2-butanone
1,3,4,6,7,8-hexahydro-4,6,6,7,8,8-hexamethylcyctopenia-γ-2-benzopyran
methyl-2-hexyl-3-oxocyclopetanedecarboxylate
3-methylcyclopentadecanone;
4-(4-Hydroxy-4-methylpentyl)cyclohex-3-enecarbaldehyde
1,12-dodecanedioic acid ethylene ester;
2-tridecenenitrile;
hexyl salicylate,
15-pentadecanolide,
2-Phenylethyl benzoate
3-Ethoxy-4-hydroxybenzaldehyde
hexadecanolide
9-cycloheptadecen-1-one
3-(5,5,6-Trimethylbicyclo[2.2.1]hept-2-yl)cyclohexan-1-ol
Pentyl-2-hydroxybenzoate,
3,7-Dimethyloctane-1,7-diol
p-cresyl phenylacetate
1-Methyl-1-(3S,8S)-1,2,3,4,5,6,7,8-octahydro-3,8-dimethylazulen-5-yl)ethyl acetate
3-Hexenyl 2-hydroxybenzoate
1,4-Dioxacycloheptadecane-5,17-dione
2,5-Dimethyl-4-hydroxy-2,3-dihydrofuran-3-one
2-Phenylethyl phenylacetate
TRASEOLIDE
4-methoxybenzyl alcohol
Benzyl o-hydroxybenzoate
2-Ethyl-3-hydroxy-4-pyrone
DECEN 1 AL 9
4-Hydroxy-3-methoxybenzaldehyde
Ethyl 2-methyl-4-oxo-6-pentylcyclohex-2-ene-1-carboxylate
4-(4-Hydroxy-3-methoxyphenyl)-2-butanone
3,7,11,15-Tetramethyl-1-hexadecen-3-ol
oxacycloheptadec-10-en-2-one
3,7-dimethyl-1,6-octadien-3-yl 3-phenyl-2-propenoate
2-Phenylethyl 2-hydroxybenzoate
Methyl 2,4-dihydroxy-3,6-dimethylbenzoate
5-Hydroxy-2-benzyl-1,3-doxan
PARADISAMIDE
   Examples of odorants having an acceleration value between 100 and 900 include:
3-phenyl butanal
3,7-dimethyl-6-octenol
2,6-dimethyl-7-octen-2-ol
6-Butyl-2,4-dimethyldihydropyrane
3,7-Dimethyl-2,6-octadienal
cyclohexyl ethyl acetate
3a,4,5,6,7,7a-Hexahydro-5-methoxy-4,7-methano-1H-indene
methyl-2-octynoate
decanal
3,7-Dimethyl-1-octen-7-ol
(Z)-1-(1-Methoxypropoxy)hex-3-ene
Nonen acid nitrile
(Z)-3,4,5,6,6-Pentamethylhept-3-en-2-one
2-Butyl-4,4,6-trimethyl-1,3-dioxane
2-Heptyltetrahydrofuran
hexyl butyrate
Ethyl octanoate
2,2,5-Trimethyl-4-hexanal dimethyl acetal
tricyclodecenyl propionate
p-cresyl acetate
2-propenyl heptanoate
2-methyl-3-(4-methoxyphenyl)propanal
Exo-1,7,7-trimethylbicyclo[2.2.1]hept-2-yl acetate
benzyl acetate
2,6-dimethyl-2-octanol
3,7-Dimethyl-2,6-octadien-1-thiol
Methyl 2-nonenoate
4-Methyl-1-oxaspiro[5.5]undecan-4-ol
2-Pentylcyclopentan-1-one
3,7-Dimethyl-1,6-octadien-3-ol
ethyl acetoacetate
Decyl methyl ether
1-Methyl-4-isopropenyl-6-cyclohexen-2-one
n-Hexyl 2-butenoate
3,7-Dimethyl-1,6-octadien-3-ol acetate
p-Menth-1-en-8-yl acetate
3,7-Dimethyloctan-3yl acetate
2-Methyl-4-propyl-1,3-oxathiene
α,3,3-Trimethylcyclohexylmethyl acetate
α,3,3-Trimethylcyclohexylmethyl formate
3-phenylpropanal
1,3,3-Trimethylbicyclo(2,2,1)heptan-2-ol
2-Pentyl-3-methyl-2-cyclopenten-1-one
3,7-Dimethyl-6-octen-3-ol
o-t-butylyl-cyclohexyl acetate
4-(1,1-Dimethylpropyl)cyclohexanone
Ethylacetoacetate ethylene glycol ketal
3-Methylene-7-methyl-1-octen-7-yl acetate
4-methylphenylacetaldehyde
3,5,5-trimethylhexl acetate
4-Methoxy-1-propenylbenzene (E)
p-Menthan-8-yl acetate
nonyl acetate
isolongifolene oxide
methyl-2-nonynoate
benzyl propionate
4-methoxyacetophenone
3,7-dimethyloctan-3-ol
1,7,7-Trimethylbicyclo(2,2,1)heptan-2-ol
3,7-Dimethyl-2-methylenocta-6-enal
phenylacetaldehyde dimethyl acetal
1-Methyl-4-isopropyl-3-cyclohexan-1-ol
ethyl 2,6,6-trimethyl-1,3cycloheadiene-1-carboxylate
2,4-Dimethyl-4-phenyltetrahydrofuran
Ethyl propanedioate
2,6-dimethyl-7-octenyl-2-yl acetate
(Z)-3,7-Dimethylocta-2,6-dienenitrile
exo-1,7,7-Trimethylbicyclo(2,2,1)hept-2-ylpropionate
cis-3,7-Dimethyl-2,6-octadien-1-yl ethanoate
3-Methyl-4-(2,6,6-trimethylcyclohex-1-enyl)out-3-en-2-one
2-isopropenyl-5-methylhex-4-enyl acetate
2,4-Dimethylcyclohexylmethyl acetate
3,5-Dimethylcyclohex-3-ene-1-methyl acetate
VERDORACINE
1-Phenylethyl propionate
2,4-Demethylcyclohex-3-ene-1-methanol
p-isopropylbenzaldehyde,
undecanal
2-ethylidene-6-isopropoxy-bicyclo[2.2.1] heptane
3-Methyl-5-propyl-2-cyclohexan-1-one
8,8-dimethyl-7-[1-methylethyl]-6,10-dioxaspiro[4,5]decane
3,7-Dimethyl-1,6-octadien-3-yl propionate
2-Methyldecanal
1,1-Dimethoxy-2-phenylpropane
o-tertiary butyl cyclohexanol
VIOLET NITRILE CI (Q)
4n-Butyl-4-hydroxybutyric acid lactone
CRESSANTHER
3,7-dimethyl-6-octen-1-yl formate
2-Phenylethyl acetate
3,7-dimethyl-6-octen-1-yl acetate
8,9-epoxy cedrane
p-isopropylcyclohexanol
2,6-dimethyl-2-octanol
4-isopropyl cyclohexanol
p-tert-Butylcyclohexl acetate
cis-6-nonenol
5-Methyl-2-(1-methylethyl)cyclohexanol
γ-methylonone
Ethyl 2,4-dimethyldioxolane-2-acetate
1-Methyl-4-isopropylcyclohexane-8-ol
JASMATONE
3,7-Dimethyl-1-octen-7-ol
cis-3-hexenyl methylbutyrate
phenylethyl formate
trans-3,7-Dimethyl-2,6-octadien-1-yl acetate
4-(2,6,6-Trimethyl-1-cyclohexen-1-yl)-3-buten-2-one
ROSSITOL
2,4-dimethyl cyclohexane methanol
cis-8-Methyl-1-oxaspiro[4.5]decan-2-one
2-Methylpent-2-en-1-oic acid
1a,3a,6a)-2′,2′,3,7,7-Pentamethylspiro(biclo[4.1.0]heptane-2,5′-[1,3]dioxane
g-nonalactone
10-undecenal
α-ionone
1-methyl-1-methoxycyclododecane
3,7-Dimethyl-1,6-octadien-3yl 2-methylpropanoate
2,2,5-trimethyl-5-pentylcyclopentanone
CUMIN NITRILE
4-Methoxybenzyl acetate
3,7-Dimethyl-1,6-nonadien-3-ol
cis-2,6-Dimethyl-2,6-octadien-8-ol
spiro[furan-2(3H), 5′-(4,7-methano-5H-indene)], decahydro
ethyl safranate
1-p-Menthen-8-ol, 1-Methyl-4-isopropyl-1-cyclohexen-8-ol
5,9-Dimethyl-4,8-decadienal
benzyl-n-butyrate
(E)-3,7-Demethyl-2,6-octadienyl 2-methylcrotonate
2-Methyl-3-phenyl-2-propanal
o-t-amyl-cyclohexanyl acetate
ROSYRANE SUPER
Octyl-2-methylpropanoate
dimethyl benzyl carbinyl acetate
3-Methyl-1,4-octalactone
2-Methyl-4-phenyl-2-butanol
2,6-Nonadienol
isobutyl phenylacetate
(R-(E)-1-(2,6,6-Trimethyl-2-cyclohexen-1-yl)pent-1-en-3-one
LEVISTAMEL
3,7-dimethyl-1,6-nonadien-3yl acetate
1-(2,4-Dimethyl-3-cyclohexenyl)-2,2-dimethylpropan-1-one
α,α-Dimethylphenethyl alcohol
(E)-1-2,4,4-Trimethyl-2-cyclohexen-1-yl)-2-buten-1-one
1-(2,6,6-Trimethyl-1-cyclohexen-1-yl)pent-1-en-3-one
2,4,6-Trimethyl-3-cyclohexene-1methanol
trans-3,7-Dimethyl-2,7-octadien-1-ol
1,1-Diethoxy-3,7-dimethyl-2,6-octadiene
1-Phenyl-4-penien-1-one
cedryl methyl ether
1-Methyl-4-isopropanylcyclohexen-3-ol
phenylethyl isoamyl ether
3-Methylene-7-methyl-1-octen-7-yl acetate
6-ethylideneoctahydro-5,8-methano-2H-benznopyran
3,7-Dimethyl-1-octanol
3,7-Dimethyl-1,6-octadien-3-yl butyrate
2-hexyl-2-cyclopenten-1-one
methoxycyclodecan
1-Cyclohexylethyl-2-butenoate
5,6-epoxy-2,6,10,10-tetramethyl bicyclo[7,2,0]undecane
Tetrahydro-4-methyl-2phenyl-2H-pyran
acetaldehyde ethyl phenylethyl acetal
trans-3,7-Dimethyl-2,6-octadien-1-yl propionate
6,10-dimethyl-5,9-undecadien-2-one
6-Methyl-2-(4-methylcyclohex-3-enyl)hept-1,5-diene
3-Methyl-2-(2-pentenyl)-2-cyclopenten-1-one isomers
2-ethoxy-9methylen-2,6,6-trimethylbicyclo[3,3,1]nonane
Tetrahydro-4-methyl-2-propyl-2H pyran-4-yl acetate
trans-3,7-Dimethyl-2,6-octadien-1-yl isobutyrate
p-Methyltetrahydroquinoline
decahydro-b-naphtyl acetate
dodecanal
1-phenylethyl alcohol
(E)-7,11-Dimethyl-3-methylenedodeca-1,6,10-diene
3-(isopropylphenyl)butanal
ethyl-2-ethyl-6,6-dimethyl-2-cyclohexene
3,7-dimethyl-2(3), 6-nonadienenitrile
6-methyl-β-ionone
7-methoxy-3,7-dimethyloctanal
(Z)-1-(2,6,6-Trimethyl-1-cyclohexen-1-yl)-2-buten-1-one
Allyl (3-methylbutoxy)acetate
4-(2,5,6,6-Tetramethyl-2-cyclohexen-1-yl)-3-buten-2-one
3-Methyl-2-butenyl benzoate
3-(4-ethyl(phenyl)-2,2-dimethylpropanal
3,5,6,6-Tetramethyl-4-methyleneheptan-2-ol
δ-1-(2,6,6-Trimethyl-3-cyclohexen-1-yl)-2-buten-1-one
ethyl tricyclo [5.2.1.02.6] decan-2-carboxylate
α-1-(2,6,6-Trimethyl-2-cyclohexen-1-yl)-2-buten-1-one
9-decenol
UNDECENE 2 NITRILE
Ethyl 2-nonynoate
3,4,4a,5,8,8a-Hexahydro-3′,7-dimethylspiro[1,4-methanonaphthalene-2-(1H), 2′-oxirane]
MARENIL
Ethyl 2,3-epoxy-3-methyl-3-phenylpropionate
3,6-Dihydro-2,4-dimethyl-6-phenyl-2H-pyran
cis-trans-2-Methyl-2-vinyl-5(2-hydroxy-2-propyl)tetrahydrofuran
4-methyl-3decenn-5-ol
Octahydro-4,7-methano-1H-indene-5-yl acetate
2-Methylundecanal
2-heptyl cyclopentanone
HERBANATE
6-sec-Butylquinoine
allyl cyclohexyloxyacetate
5-phenyl-5-methyl-3-hexanone
DISPIRONE
BOURGEONAL
3,7-Dimethyl-6-octen-1-yl propanoate
phenylethyl isobutyrate
1,2,3,4,5,6,7,8-Octahydro-8,8-dimethyl-2-naphthaldehyde
1-(5,5-Dimethyl-1cyclohexen-1-yl)pent-4-en-1-one
Methyl 2-hydroxybenzoate
ELINTAAL Forte
allyl cyclohexyl propionate
3,7-Dimethyl-6-octen-1-yl 2-methylpropanoate
INDOCLEAR
AZARBRE
2-Phenoxyethyl propionate;
Ethyl 2-methoxybenzoate
3-Phenyl-2-propanal
2,2-Dimethyl-3-(p-ethylphenylpropanal
2,7-Dimethyl-10-(1-methylethyl)-1-oxaspiro[4,5]deca-3,6-diene
1,3,4,6,7,8a-1,1,5,5-tetramethyl-2H-2,4a-methanonaphthalen-8)5H)-one
5-methyl-3-heptanone oxime
cis-3-hexenyl benzoate
2,3,4,5,6,7,8-Octahydro-8,8-dimethyl-2-naphthaldehyde
5-Hydroxyundecanoic acid lactone
4-methoxybenzaldehyde
4-methyl-3-decen-5-ol
4-n-Hexyl-4-hydroxybutanoic acid lactone
Allyl (2-methylbutoxy)acetate
p-Mentha-8-thio-3-one
dodecahydro-3a,6,6,9a-tetramethylnaphto(2,1-b)-furan
5-methyl-3-heptanone oxime
4-(1-ethoxyvinyl)-3,5,5,5-tetramethylcyclo-hexanone
2-(4-tert-Butylbenzyl)propionaldehyde
Cyclohexyl lactone
decanol
1-(2,6,6-Trimethylcyclohexa-1,3-denyl)-2-buten-1-one
2-methyl-3-(4-isopropylphenyl)propanal
1-(4-ISOPROPYLCYCLOHEXYL)-ETHANOL

### Odorant's Hedonic Contributions

It is also important to construct the fragrance with a balanced olfactive intensity in order not to overwhelm the consumer or to be aesthetically unappealing. Constructing each segment for the targeted application or intended effect must be based on balanced impact in accordance to odor detection threshold values (ODT) while at the same time answering to certain physico-kinetic rules to give a well-rounded experience to the consumer.

Upon their release in headspace, odorants are detected based on their odor detection threshold values. Odor detection thresholds are defined as the lowest concentration of odorants in a selected medium (air or water) to be detected. By including odor values of odorants in the model, one can further improve on the values for predicted performance of once odorants are released from the partition into the air.

Various databases for experimental odor detection threshold values in various partitions such as water and air are available. See Compilation of Odor and Taste Threshold Values Data, American Society for Testing and Materials, F. A. Fazzalari Editor; Booleans Aroma Chemical Information Service (BACIS)).

In order to create a linear citrus fragrance upon dilution, the authors further hedonically define each kinetic “water release group” based on the odor detection threshold values and concentration of its composing odorants along with their odor descriptors as defined by a panel of expert perfumers. Once the odorants are grouped in a “water release group” based on their Ω values, their hedonic contribution is estimated using the following equation:

$$Odor Impact = \frac{Parts in formula}{O D T} \qquad [ 5 ]$$

Within each water release group, the odor contributions for each composing odorant are then added to calculate the overall odor contribution of each “water release group”. This not only provides the person skilled in the art with the capability of estimating the odor intensity of each “water release group” but also the hedonic bloom contribution of each odorant within the “water release group”. A simple percentage calculation can then be performed to obtain the percent contribution of each odorant within the “water release group” as shown below:

$${( \% odor contribution )}_{odorant} = \frac{{( Odor Impact )}_{odorant}}{{( Total Odor Impact )}_{water release group}} \qquad [ 6 ]$$

Odorants are described according to a classification given by a panel of expert perfumers. The odorants composing each water release group is defined hedonically according to two descriptors given by the panelists. For example, odorants are defined as green if either one of the two descriptors contains a “green” or “grass” definition as shown below:

| Odor Descriptor 1 | Odor Descriptor 2 |
|---|---|
| Green | Cucumber |
| Green | Cuminic |
| Green | Earthy |
| Green | Fatty/Greasy |
| Green | Floral |
| Green | Fruity |
| Green | Citrus |
| Green | Hay |
| Green | Herbal |
| Green | Honeysuckle |
| Green | Hyacinth |
| Green | Lavender |
| Green | Leaf |
| Green | Lilac |
| Green | Metallic |
| Green | Mushroom |
| Green | Musty |
| Green | Narcissus |
| Green | Nutty |
| Green | Pine |
| Green | Rose |
| Green | Violet |
| Grass | Green |
| Grass | Fruity |
| Grass | Violet |
| Grass | Green |
| Grass | Fruity |
| Aldehydic | Green |
| Amber/Woody | Green |
| Aniseed | Green |
| Apple | Green |
| Balsamic | Green |
| Blackcurrant | Green |
| Citrus | Green |
| Earthy | Green |
| Tropical | Green |
| Fruity | Green |
| Galbanum | Green |
| Hyacinth | Green |
| Jamin | Green |
| Leather | Green |
| Marine | Green |
| Mimosa | Green |
| Muguet | Green |
| Mushroom | Green |
| Narcissus | Green |
| Pine | Green |
| Rose | Green |
| Sandalwood | Green |
| Tuberose | Green |
| Violet | Green |
| Woody | Green |

Odorants are described as either citrus, floral, fruity or green based on odor definitions in contained in either Descriptor 1 or Descriptor 2 but more preferably, based on attributes found in Descriptor 1.

### APPLIED PERFUME EXAMPLES

As an illustration, linear citrus fragrances for rinse-off were designed according to the rationale described in the invention to fit the application needs of three different wash-off categories: dish-washing and surface cleaners, body wash and shampoos, conditioners, and finally laundry detergents.

Examples of perfumes engineered for linear citrus, citrus/fruity, citrus/floral and citrus/green release during rinse-off are shown in the following examples.

### I. Linear Citrus

The following general examples are to illustrate linear citrus release during rinse-off conditions.

### A. Dish Washing and Surface Cleaners

The fragrance designed for these types of application are intended to give a superior impact to the consumer whilst avoiding any hedonics or streak residual on the targeted cleaned surface. One can design a pleasant and full experience for the user of the market product with the engineered perfume while at the same time minimizing substantivity.

Formulations for these types of household and/or industrial applications must contain perfumes that answer to the following criteria: at least 20%, preferably at least 30% of the odorant constituents must have γ values characteristic of flash release in aqueous dilutions, as described above (γ≧900).

In addition to the required content of flash release odorants mentioned above, the percentage of delayed release (or deposition) odorants must not exceed 30%, preferably not exceed 15% of the perfume's total content.

In order to have an impactful citrus released background, at least three, preferably four of the water release groups constructed based on odorants' Ω values must have at least 30%, preferably at least 40% of their overall odor contributed by one or more citrus odorants.

As an illustrative example, a fragrance (“Flash Release Type Citrus”) was designed to give maximum linear citrus impact during rinse-off with minimal deposition on targeted surface. The perfume is shown below:

TABLE 9
Flash Release Type Citrus

| | | | | | Odor | % Odor |
|---|---|---|---|---|---|---|
| | parts | γ | Ω | ODT (ppb) | Descriptor | Contribution |
| Water Release Group 1 | | | | | | |
| d-LIMONINE | 50.00 | 8200.7592 | 586.22897 | 430 | CITRUS | 99.40 |
| ETHYL 2-METHYL BUTYRATE | 0.30 | 14512.2887 | 21.185694 | 20 | APPLE | 0.60 |
| total parts | 50.30 | | | | % Citrus Odor | 99.40 |
| Water Release Group 2 | | | | | | |
| HEXYL ACETATE | 0.90 | 3115.7849 | 1.3050809 | 950 | FRUITY | 2.18 |
| DIHYDROMYRCENOL | 20.00 | 844.4125 | 0.2646234 | 810 | CITRUS | 56.73 |
| CIS-3-HEXENYL ACETATE | 0.90 | 1364.2710 | 0.1522188 | 170 | GREEN | 12.16 |
| ETHYL ACETOACETATE | 0.50 | 840.3492 | 0.1081678 | 54 | APPLE | 21.27 |
| CIS-3-HEXEN-1-OL | 0.30 | 1589.1101 | 0.0789638 | 90 | GREEN | 7.66 |
| total parts | 22.60 | | | | % Citrus Odor | 56.73 |
| Water Release Group 3 | | | | | | |
| CITRONELLYL NITRILE | 1.40 | 913.0422 | 0.0881181 | 71 | CITRUS | 17.38 |
| APPLINAL | 1.40 | 554.7882 | 0.0485108 | 55 | APPLE | 22.43 |
| TETRAHYDROLINALOOL | 5.40 | 503.4877 | 0.0151079 | 380 | FLORAL | 12.52 |
| ROSSITOL | 2.90 | 303.8040 | 0.0182541 | 440 | CITRUS | 5.81 |
| ETHYL LINALOOL | 5.70 | 275.6310 | 0.0121484 | 120 | CITRUS | 41.88 |
| total parts | 16.80 | | | | % Citrus Odor | 65.04 |
| Water Release Group 4 | | | | | | |
| OXANE | 0.06 | 810.1552 | 0.0019673 | 58 | CITRUS | 0.48 |
| GARDAMIDE | 5.00 | 88.4744 | 0.0019517 | 24 | CITRUS | 94.48 |
| ALLYL CYCLOHXYL PROPIONATE | 0.90 | 126.7982 | 0.0008514 | 81 | FRUITY | 5.04 |
| total parts | 5.96 | | | | % Citrus Odor | 94.96 |
| Water Release Group 5 | | | | | | |
| MEFRANAL | 0.30 | 84.7562 | 0.0000885 | 17 | CITRUS | 100.00 |
| total parts | 0.30 | | | | % Citrus Odor | 100.00 |
| Water Release Group 6 | | | | | | |
| METHYL DIHYDRO JASMONATE | 1.60 | 8.3984 | 0.00000381 | 0.23 | FLORAL | 95.82 |
| EBANOL | 0.14 | 15.5977 | 0.00000108 | 54 | SANDALWOOD | 0.04 |
| CIS-3-HEXENYL SALICYLATE | 0.60 | 2.8507 | 0.00000015 | 1.9 | GREEN | 4.34 |
| total parts | 2.34 | | | | % Citrus Odor | 0.00 |
| DIPROPYLENE GLYCOL | 1.70 | | | | | |
| total perfume parts | 100.00 | | | | | |

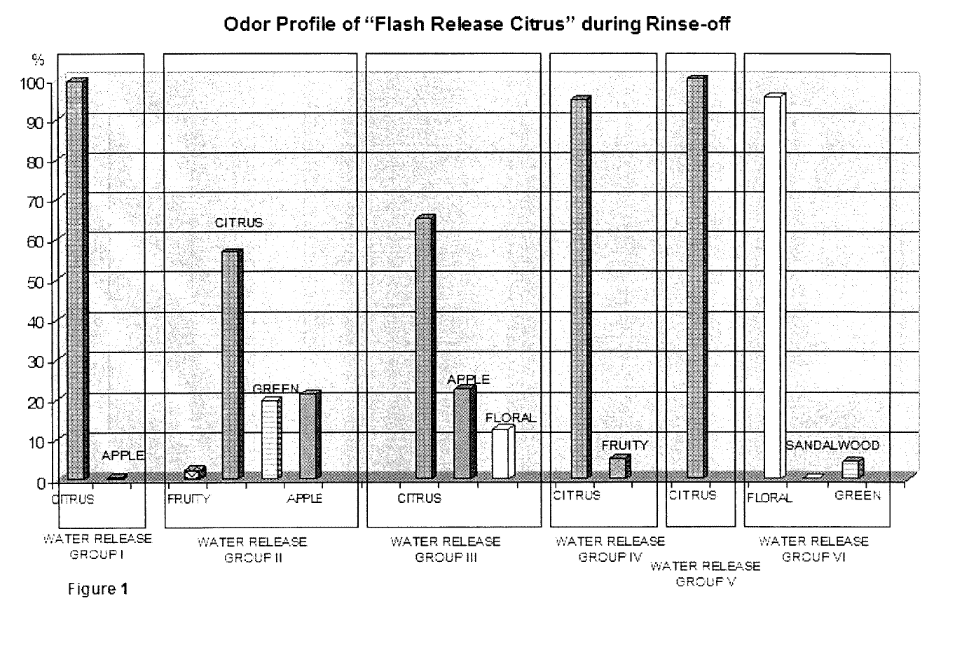

The odor profile of each water release group is expressed in percentage contribution according to odor type. This kinetic odor progression of the perfume in rinse-off conditions is shown in FIG. 1.

The perfume odorants determined by the inventors to result in flash release in water dilutions are: d-limonene, ethyl 2-methylbutyrate, hexyl acetate, cis-3-hexenol, cis-3-hexenyl acetate, and citronellyl nitrile. The fragrance odorants' physico-kinetics properties are as follow:

TABLE 10

| | parts | γ |
|---|---|---|
| Flash Release Odorants | | |
| ETHYL 2-METHYL BUTYRATE | 0.30 | 14612.2887 |
| d-LIMONENE | 50.00 | 8200.7592 |
| HEXYL ACETATE | 0.90 | 3118.7849 |
| CIS-3-HEXEN-1-OL | 0.30 | 1569.1101 |
| CIS-3-HEXENYL ACETATE | 0.90 | 1384.2710 |
| CITRONELLYL NITRILE | 1.40 | 913.0422 |
| total | 53.80 | |
| Sustained Release Odorants | | |
| DIHYDROMYRCENOL | 20.00 | 644.4128 |
| ETHYL ACETOACETATE | 0.50 | 640.3492 |
| OXANE | 0.06 | 610.1552 |
| APPLINAL | 1.40 | 554.7882 |
| TETRAHYDOLINALOOL | 5.40 | 503.4877 |
| ROSSITOL | 2.90 | 303.8040 |
| ETHYL LINALOOL | 5.70 | 275.6310 |
| ALLYL CYCLOHEXYL PROPIONATE | 0.90 | 126.7982 |
| total | 36.86 | |
| Delayed Release Odorants | | |
| MEFRANAL | 0.30 | 84.7562 |
| GARDAMIDE | 5.00 | 66.4744 |
| EBANOL | 0.14 | 15.5977 |
| METHYL DIHYDRO JASMONATE | 1.60 | 8.3964 |
| CIS-3-HEXENYL SALICYLATE | 0.60 | 2.8007 |
| total | 7.64 | |
| DIPROPYLENE GLYCOL | 1.70 | |
| total perfume parts | 100.00 | |

These flash release odorants and the deposition odorants (also referred to as delayed release odorants) are calculated to make up respectively 54% and 8% of the total perfume.

The above perfume (Flash Release Citrus Type) provides a released citrus linear hedonic impact in use while also leaving a minimum amount of residual fragrance or streaks upon completing the cycle or the cleaning experience.

### B. Body-Wash, Soap, Shampoo and Conditioners

It is important to establish that a perfume during a wash off experience in household, cosmetic and industrial applications such as body wash, shampoo, conditioners etc. must provide a well rounded, impactful hedonic experience that will last throughout the entire rinsing process. In most instances, the performance attributes of the product are largely dependent on the impact, intensity and overall hedonic quality of its perfume in use. For instance, consumers often base their liking of the product to a diffuser-type of fragrance release in use. In other words, a long sustained perfume residence profile during and after use in an enclosed are (bathroom, shower room etc.).

Residence time of the chosen odorants within the perfume formula must therefore be optimally based on their acceleration γ values out of the water partition. Since γ is derived partly based on the vapor pressure and the diffusion coefficients in water as well as in the vapor phase, it is an indication of the residence time of odorants.

Grouping odorants in a perfume according to their mass correlated water release values and optimizing specific release groups will serve to result in a longer residence time in headspace and a more rounded hedonic experience for the user during the wash-off.

Rinse-off experience of wash-off systems such as shampoo, conditioners, body wash etc. should provide the consumer with a sustained hedonic release.

Perfumes for wash-off systems such as shampoos, conditioners and body-wash lotions and gels must have at least 30%, preferably at least 40% of the total perfume with γ values between 900 and 100, as defined earlier within this patent.

In addition, linear citrus release can be engineered based on odorants' odor detection threshold values and concentration within a perfume. Using methods described earlier, at least three, preferably four release groups defined by Ω values of their composing odorants must have an overall citrus odor value of at least 30%, preferably 40% of the overall release group odor from one or more odorants within the release group.

Below in Table 11 is an illustrative example (“Citrus Sustained Release-Type”) of a fragrance engineered for sustained release of a citrus note in high water dilutions.

TABLE 11
Citrus Sustained Release Type Fragrance

| | | | | | Odor | % Odor |
|---|---|---|---|---|---|---|
| | Parts | γ | Ω | odt | Descriptor | Contribution |
| Water Release Group 1 | | | | | | |
| d-LIMONINE | 12.00 | 8200.7592 | 154.881433937 | 430.00 | CITRUS | 85.04 |
| ETHYL 2-METHYL BUTYRATE | 0.30 | 12827.5826 | 18.784383082 | 20.00 | FRUITY | 34.98 |
| | | | | | % Citrus Odor | 65.04 |
| Water Release Group 2 | | | | | | |
| LIGUSTRAL | 4.90 | 1704.6486 | 1.537120485 | 110.00 | GREEN | 7.01 |
| DIHYDROMYRCENOL | 25.00 | 865.6450 | 1.538238177 | 819.00 | CITRUS | 4.85 |
| HEXYL ACETATE | 0.50 | 3118.7849 | 1.305060933 | 550.00 | FRUITY | 0.15 |
| CITRONELLAL | 5.00 | 1345.8902 | 0.838905732 | 33.00 | CITRUS | 23.84 |
| CITRONELLOL | 5.00 | 865.5602 | 0.289877934 | 29.00 | FLORAL | 27.12 |
| CIS-3-HEXENYL ACETATE | 0.90 | 1384.2710 | 0.182218814 | 170.00 | GREEN | 0.83 |
| CITRAL | 2.50 | 857.0901 | 0.133615808 | 12.00 | CITRUS | 32.77 |
| TETRAHYDROLINALOOL | 7.00 | 503.4877 | 0.117505958 | 380.00 | FLORAL | 2.96 |
| CIS-3-HEXEN-1-OL | 0.30 | 1569.1101 | 0.075983597 | 90.00 | CITRUS | 0.52 |
| | | | | | % Citrus Odor | 81.99 |
| Water Release Group 3 | | | | | | |
| CITRONELLYL NITRILE | 1.40 | 913.0422 | 0.065115111 | 71.00 | CITRUS | 19.46 |
| ROSSITOL | 10.00 | 303.8040 | 0.083048652 | 440.00 | CITRUS | 22.45 |
| APPLINAL | 1.40 | 554.7882 | 0.048510807 | 55.00 | FRUITY | 25.14 |
| RHUBAFURAN | 1.50 | 475.0599 | 0.025835530 | 440.00 | CITRUS/GREEN | 3.37 |
| ETHYL ACETOACETATE | 0.50 | 540.3492 | 0.023075908 | 55.00 | FRUITY | 8.98 |
| ETHYL LINALOOL | 2.50 | 275.6310 | 0.010472748 | 120.00 | CITRUS | 20.58 |
| | | | | | % Citrus Odor | 85.58 |
| Water Release Group 4 | | | | | | |
| OXANE | 0.08 | 510.1652 | 0.001957274 | 58.00 | CITRUS | 0.49 |
| GARDAMIDE | 5.00 | 65.4744 | 0.001051679 | 24.00 | CITRUS | 94.48 |
| ALLYL CYCLOHXYL PROPIONATE | 0.90 | 128.7982 | 0.000851449 | 81.00 | FRUITY | 5.04 |
| | | | | | % Citrus Odor | 94.96 |
| Water Release Group 5 | | | | | | |
| MEFRANAL | 1.50 | 84.7562 | 0.000443184 | 17.00 | CITRUS | 100.00 |
| | | | | | % Citrus Odor | 100.00 |
| Water Release Group 6 | | | | | | |
| METHYL DIHYDRO JASMONATE | 5.00 | 8.3964 | 0.000012010 | 0.23 | FLORAL | 90.17 |
| CIS-3-HEXENYL SALICYLATE | 4.50 | 2.5007 | 0.000001122 | 1.90 | FLORAL | 9.52 |
| EBANOL | 0.14 | 15.5977 | 0.000001077 | 54.00 | SANDALWOOD | 0.81 |
| | | | | | % Citrus Odor | 0.00 |
| DIPROPYLENE GLYCOL | 1.80 | | | | | |
| total perfume parts | 100.00 | | | | | |

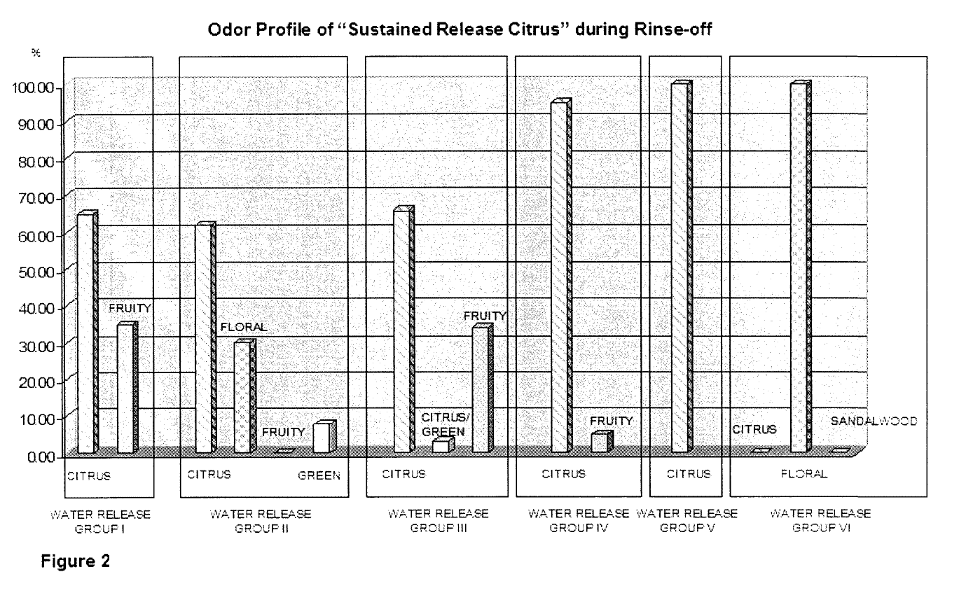

The odor profile of each water release group is expressed in percentage contribution according to odor type. This kinetic odor progression of the perfume in rinse-off conditions is shown in FIG. 2

The perfume odorants determined by the inventors to result in sustained release in water dilutions are: citronellol, dihydromyrcenol, citral, ethyl acetoacetate, oxane, applinal, tetrahydrolinalool, rhubafuran, rossitol, ethyl linalool, allyl cyclohexyl propionate. The physico-kinetic properties of the perfume composing odorants are as follow:

TABLE 12

| | parts | γ |
|---|---|---|
| Flash Release Odorants | | |
| ETHYL 2-METHYL BUTYRATE | 0.30 | 12827.5626 |
| d-LIMONENE | 12.00 | 8200.7592 |
| HEXYL ACETATE | 0.90 | 3118.7849 |
| LIGUSTRAL | 4.90 | 1704.6486 |
| CIS-3-HEXEN-1-OL | 0.30 | 1569.1101 |
| CIS-3-HEXENYL ACETATE | 0.90 | 1384.2710 |
| CITRONELLAL | 5.00 | 1345.0902 |
| CITRONELLYL NITRILE | 1.40 | 913.0422 |
| Total | 25.70 | |
| Sustained Release Odorants | | |
| CITRONELLOL | 5.00 | 866.5602 |
| DIHYDROMYRCENOL | 25.00 | 866.5450 |
| CITRAL | 2.50 | 857.0901 |
| ETHYL ACETOACETATE | 0.50 | 640.3492 |
| OXANE | 0.06 | 610.1552 |
| APPLINAL | 1.40 | 554.7882 |
| TETRAHYDOLINALOOL | 7.00 | 503.4877 |
| RHUBAFURAN | 1.50 | 476.0599 |
| ROSSITOL | 10.00 | 303.6040 |
| ETHYL LINALOOL | 2.50 | 275.6310 |
| ALLYL CYCLOHEXYL PROPIONATE | 0.90 | 126.7982 |
| Total | 56.36 | |
| Delayed Release Odorants | | |
| MEFRANAL | 1.50 | 84.7562 |
| GARDAMIDE | 5.00 | 66.4744 |
| EBANOL | 0.14 | 15.5977 |
| METHYL DIHYDRO JASMONATE | 5.00 | 8.3964 |
| CIS-3-HEXENYL SALICYLATE | 4.50 | 2.8007 |
| Total | 16.14 | |
| DIPROPYLENE GLYCOL | 1.80 | |
| total perfume parts | 100.00 | |

The above perfume provides a linear sustained release citrus hedonic impact during the process of rinse-off in formulations such as shampoo, conditioners and body wash amongst others.

### C. Laundry Products

At the end of a typical wash cycle, perfume deposition is often minimal due to the relative solubility and water-release values of a number of odorants making up a typical perfume in addition to the large amount of water used during a typical household wash cycle whether automated or manual. It is therefore important to engineer fragrances with maximum deposition on woven and non-woven surfaces for obvious commercial and environmental reasons when considering these types of household and industrial applications.

Furthermore, many parts of the world still rely on hand-washing of laundry rather than using automated appliances as often found in Western countries. It is therefore important to provide the consumer with an agreeable impactful hedonic experience during the wash-off whilst also resulting with a significant amount of fragrance deposition on the woven and non-woven surfaces at the end of the process.

Since water release values are derived based on activity and water diffusion coefficients of odorants in water, as well as partition energies of these odorants for polar and non polar partitions, vapor pressure etc., it is possible to predict quantitatively the substantivity of the individual odorants considered in the perfume in water.

Based on the Ω values of odorants and their subsequent grouping in various release groups as shown in methods above, this invention provides a person skilled in the art with the possibility to engineer the release of a citrus hedonic note or perfumes to be perceived by the consumer during a manual or automated laundry cycle. In addition to a linear release of a citrus hedonic note, the method mentioned in this invention will allow a significant amount of fragrance to be deposited on the woven and non-woven surfaces upon completion of the wash cycle. In addition, fragrances designed according to methods described herein for laundry applications will limit unnecessary environmental waste of perfumes.

Perfumes intended for maximum deposition in wash-off systems must have at least 40% and preferably at least 50% of the total perfume with delayed release type of odorants (depositors) as defined in the herein invention.

In addition to criteria for maximum deposition of perfume defined above, at least three, preferably four of the water release groups constructed based on odorants' Ω values must have at least 30%, preferably at least 40% of their overall odor contributed by one or more citrus odorants. These fragrances will therefore also provide the consumer with a perception of linear sustained citrus perfume throughout the process of rinse-off.

A perfume (Deposition/Linear Release Citrus Perfume) for laundry detergents designed to provide maximum deposition of fragrance as well as a linear release of a citrus note during the process of rinse-off is shown below in Table 13.

TABLE 13

| | Parts | γ | φ | φ · γ | Ω |
|---|---|---|---|---|---|
| Water Release Group 1 | | | | | |
| d-LIMONINE | 3.50 | 8200.7592 | 0.001571820 | 12.896119495 | 45.115418232 |
| ETHYL 2-METHYL BUTYRATE | 0.25 | 12027.5828 | 0.004361536 | 55.947878874 | 13.958569218 |
| Water Release Group 2 | | | | | |
| HEXYL ACETATE | 1.48 | 3118.7849 | 0.000454948 | 1.450067703 | 2.140498258 |
| LIGUSTRAL | 0.82 | 1704.6488 | 0.000195997 | 0.334108221 | 0.273992309 |
| DIHYDROMYRCENOL | 3.28 | 886.5450 | 0.000073316 | 0.083531527 | 0.208402502 |
| CITRONELLAL | 0.62 | 1345.0902 | 0.000124439 | 0.187331348 | 0.137285332 |
| CITRONELLOL | 1.23 | 888.5802 | 0.000066703 | 0.057935587 | 0.171287328 |
| Water Release Group 3 | | | | | |
| ALDEHYDE C11 | 4.10 | 420.7513 | 0.000026178 | 0.011014407 | 0.045183224 |
| CIS-3-HEXENYL ACETATE | 0.25 | 1384.2710 | 0.000130208 | 0.180243127 | 0.044343889 |
| RHUBAFURAN | 2.46 | 476.0589 | 0.000037580 | 0.017890353 | 0.044014318 |
| TETRAHYDROLINALOOL | 2.46 | 503.4877 | 0.000033341 | 0.018788566 | 0.041298751 |
| CITRONELLYL NITRILE | 0.82 | 913.8422 | 0.000053290 | 0.048655794 | 0.039901422 |
| ROSSITOL | 4.10 | 303.8040 | 0.000020753 | 0.005304865 | 0.025852326 |
| CIS-3-HEXEN-1-OL | 0.08 | 1569.1101 | 0.000163540 | 0.258611990 | 0.021044119 |
| ETHYL ACETOACETATE | 0.25 | 640.3492 | 0.000072026 | 0.048159815 | 0.011358369 |
| ETHYL LINALOOL | 2.46 | 275.8310 | 0.000015196 | 0.004189096 | 0.010306130 |
| ALLYL CYCLOHEXYL PROPIONATE | 9.02 | 126.7962 | 0.000007481 | 0.000946055 | 0.006534198 |
| APPLINAL | 0.25 | 554.7832 | 0.000052457 | 0.034850577 | 0.008524626 |
| Water Release Group 4 | | | | | |
| MEFRANAL | 12.30 | 84.7562 | 0.000003468 | 0.000295458 | 0.003834447 |
| GARDAMIDE | 16.40 | 86.4744 | 0.000003164 | 0.000210338 | 0.003449823 |
| OXANE | 0.10 | 810.1562 | 0.000053737 | 0.032787903 | 0.003228827 |
| MANDARIN ALDEHYDE | 8.20 | 90.2674 | 0.000003454 | 0.000311846 | 0.002557369 |
| CITRAL | 0.03 | 857.0801 | 0.000062358 | 0.053448322 | 0.001753201 |
| EMPETAL | 2.48 | 95.5058 | 0.000004462 | 0.000433836 | 0.001057335 |
| Water Release Group 5 | | | | | |
| LAURONITRILE | 4.10 | 52.2181 | 0.000001569 | 0.000081912 | 0.000135870 |
| Water Release Group 6 | | | | | |
| METHYL DIHYDRO JASMONATE | 8.20 | 3.3054 | 0.000000285 | 0.000002402 | 0.000015850 |
| PARADISAMIDE | 2.90 | 7.7893 | 0.000000242 | 0.000001880 | 0.000005453 |
| EBANOL | 0.30 | 15.5977 | 0.000000492 | 0.000007590 | 0.000002505 |
| CIS-3-HEXENYL SALICYLATE | 7.38 | 2.8007 | 0.000000068 | 0.000006248 | 0.000001841 |
| | 100.00 | | | | |

| | | Odor | % Odor |
|---|---|---|---|
| | odt | Descriptor | Contribution |
| Water Release Group 1 | | | |
| d-LIMONINE | 430.00 | CITRUS | 39.44 |
| ETHYL 2-METHYL BUTYRATE | 20.00 | FRUITY | 80.58 |
| | | % Citrus Odor | 39.44 |
| Water Release Group 2 | | | |
| HEXYL ACETATE | 950.00 | FRUITY | 1.93 |
| LIGUSTRAL | 110.00 | GREEN | 9.26 |
| DIHYDROMYRCENOL | 810.00 | CITRUS | 5.04 |
| CITRONELLAL | 33.00 | CITRUS | 30.54 |
| CITRONELLOL | 29.00 | FLORAL | 52.01 |
| | | % Citrus Odor | 36.98 |
| Water Release Group 3 | | | |
| ALDEHYDE C11 | 7.70 | CITRUS | 75.15 |
| CIS-3-HEXENYL ACETATE | 170.00 | GREEN | 0.20 |
| RHUBAFURAN | 440.00 | CITRUS | 0.79 |
| TETRAHYDROLINALOOL | 380.00 | FLORAL | 0.91 |
| CITRONELLYL NITRILE | 71.00 | CITRUS | 1.83 |
| ROSSITOL | 440.00 | CITRUS | 1.52 |
| CIS-3-HEXEN-1-OL | 90.00 | GREEN | 0.13 |
| ETHYL ACETOACETATE | 55.00 | FRUITY | 0.63 |
| ETHYL LINALOOL | 120.00 | CITRUS | 2.85 |
| ALLYL CYCLOHEXYL PROPIONATE | 61.00 | FRUITY | 15.72 |
| APPLINAL | 55.00 | FRUITY | 0.83 |
| | | % Citrus Odor | 81.77 |
| Water Release Group 4 | | | |
| MEFRANAL | 17.00 | CITRUS | 10.43 |
| GARDAMIDE | 24.00 | CITRUS | 9.90 |
| OXANE | 58.00 | CITRUS | 0.03 |
| MANDARIN ALDEHYDE | 1.50 | CITRUS | 79.18 |
| CITRAL | 12.00 | CITRUS | 0.04 |
| EMPETAL | 6.00 | CITRUS | 0.40 |
| | | % Citrus Odor | 100.00 |
| Water Release Group 5 | | | |
| LAURONITRILE | 6.70 | CITRUS | 100.00 |
| | | % Citrus Odor | 100.00 |
| Water Release Group 6 | | | |
| METHYL DIHYDRO JASMONATE | 0.23 | FLORAL | 80.34 |
| PARADISAMIDE | 0.80 | CITRUS | 10.89 |
| EBANOL | 54.00 | SANDALWOOD | 0.01 |
| CIS-3-HEXENYL SALICYLATE | 1.80 | FLORAL | 8.75 |
| | | % Citrus Odor | 10.89 |

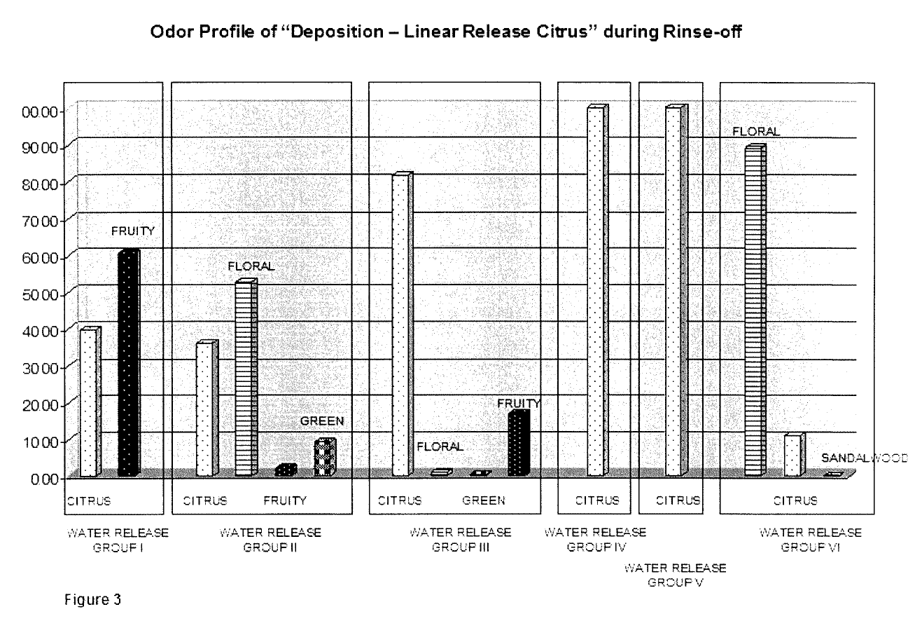

The odor profile of each water release group is expressed in percentage contribution according to odor type. This kinetic odor progression of the perfume in rinse-off conditions is shown in FIG. 3:

A total of 62% of the above perfume is composed of delayed-release odorants (also equivalent to surface depositing odorants) in high water dilutions as calculated using these odorants' γ values. These delayed release odorants are: empetal, mandarin aldehyde, mefranal, gardamide, lauronitrile, ebanol, methyl dihydro jasmonate, paradisamide and cis-3-hexenyl salicylate.

The perfume's odorants' physico-kinetic properties are shown below in table 14:

| | parts | γ |
|---|---|---|
| Flash release Odorants | | |
| ETHYL 2-METHYL BUTYRATE | 0.49 | 12827.5626 |
| d-LIMONENE | 3.28 | 8200.7592 |
| HEXYL ACETATE | 1.48 | 3118.7849 |
| LIGUSTRAL | 0.82 | 1704.6486 |
| CIS-3-HEXEN-1-OL | 0.08 | 1589.1101 |
| CIS-3-HEXENYL ACETATE | 0.25 | 1384.2710 |
| CITRONELLAL | 0.82 | 1345.0902 |
| CITRONELLYL NITRILE | 0.82 | 913.0422 |
| total | 8.04 | |
| Sustained Release Odorants | | |
| CITRONELLOL | 1.23 | 868.5602 |
| DIHYDROMYRCENOL | 3.28 | 866.5450 |
| CITRAL | 0.03 | 857.0901 |
| ETHYL ACETOACETATE | 0.25 | 640.3492 |
| OXANE | 0.10 | 610.1552 |
| APPLINAL | 0.25 | 544.7882 |
| TETRAHYDOLINALOOL | 2.46 | 503.4877 |
| RHUBAFURAN | 2.46 | 476.0599 |
| ALDEHYDE C11 | 4.10 | 420.7513 |
| ROSSITOL | 4.10 | 303.8040 |
| ETHYL LINALOOL | 2.46 | 275.6310 |
| ALLYL CYCLOHEXYL PROPIONATE | 9.02 | 126.7982 |
| total | 29.74 | |
| Delayed Release Odorants | | |
| EMPETAL | 2.46 | 96.8058 |
| MANDARIN ALDEHYDE | 8.20 | 90.2874 |
| MEFRANAL | 12.30 | 64.7562 |
| GARDAMIDE | 16.40 | 66.4744 |
| LAURONITRILE | 4.10 | 52.2161 |
| EBANOL | 0.23 | 15.5977 |
| METHYL DIHYDRO JASMONATE | 8.20 | 8.3964 |
| PARADISAMIDE | 2.95 | 7.7693 |
| CIS-3-HEXENYL SALICYLATE | 7.38 | 2.8007 |
| total | 62.23 | |

The above description is for the purposes of teaching the person of ordinary skill in the art how to practice the present invention, and it is not intended to detail all those obvious modifications and variations of it which will become apparent to the skilled worker upon reading the description.

The next examples are to illustrate perfumes intended to result in a impactful, prominent linear citrus odor during the process of rinse-off while also introducing linear release of a less dominating nuance of either one of the following hedonic groups: fruit, floral and green.

### Citrus-Green Release Perfumes

As an illustration, a citrus-green type fragrance was designed according to the rationale described in the invention to fit the application needs of three different wash-off categories: dish-washing and surface cleaners, body wash and shampoos, conditioners, and finally laundry detergents. The perfume is intended to result in a linear release of a citrus and/or green odor during rinse-off conditions.

### A. Dish Washing and Surface Cleaners

The fragrance designed for these types of application are intended to give a superior impact to the consumer whilst avoiding any hedonics or streak residual on the targeted cleaned surface. One can design a pleasant and full experience for the user of the market product with the engineered perfume while at the same time minimizing substantivity. Fragrances for this type of application are based on the physico-chemical rationale used in the preceding illustrative example: at least 30%, preferably at least 40% of the total perfume with y values between 900 and 100 coupled with the percentage of delayed release (or deposition) odorants must not exceed 30%, preferably not exceed 15% of the perfume's total content

In order to have an impactful citrus perfume released predominantly, at least three, preferably four of the water release groups constructed based on odorants' Ω values must have at least 30%, preferably at least 40% of their overall odor contributed by one or more citrus odorants.

In addition a linear release of a secondary green nuance along with the predominant citrus release may be built by having at least three, preferably four of the water release groups constructed based on odorants' Ω values must have at least 20%, of their overall odor contributed by one or more green odorants.

As an illustrative example, a flash release fragrance (“Flash Release Citrus-Cucumber”) was designed to give maximum linear citrus impact with a linear cucumber nuance during rinse-off with minimal deposition on targeted surface. The perfume is shown below in table 15. Odorants are grouped in water release groups according to their water release values. They are further characterized based on their odor descriptors and subsequently their contribution to each of the release groups' total odor.

TABLE 15
“Flash Release Type Citrus - Cucumber”

| | | Odor | Odor | | |
|---|---|---|---|---|---|
| | Parts | Descriptor 1 | Descriptor 2 | γ | φ |
| Water Release Group 1 | | | | | |
| d-LIMONINE | 42.2500 | CITRUS | ORANGE | 8200.75 | 0.001571820 |
| Water Release Group 2 | | | | | |
| STRALLYL ACETATE | 1.5000 | GREEN | FRUITY | 2576.59 | 0.000383964 |
| DIHYDRO MYRCENOL | 15.0000 | CITRUS | METALLIC | 868.55 | 0.000073318 |
| MELONAL | 0.5000 | GREEN | ALDEHYDIC | 2555.52 | 0.000309918 |
| LIGUSTRAL | 0.7700 | GREEN | LEAF | 1764.55 | 0.000196597 |
| CITRONELLAL | 1.5000 | CITRUS | CITRONELLA | 1345.08 | 0.000124439 |
| CITRONELLYL NITRILE | 4.5000 | CITRUS | NITRILE | 913.04 | 0.000053290 |
| CIS 3 HEXENYL ACETATE | 0.5000 | GRASS | FRUITY | 1384.27 | 0.000138208 |
| LINALOOL | 2.5000 | LINALOOL | | 644.41 | 0.000047749 |
| ALDEHYDE C 8 (OCTANAL) | 0.0450 | ALDEHYDIC | CITRUS | 3630.08 | 0.000455096 |
| ALDEHYDE C 10 (DECANAL) | 1.4300 | ALDEHYDIC | ORANGE | 818.28 | 0.000080573 |
| Water Release Group 3 | | | | | |
| RHUBAFURAN | 3.0000 | GREEN | GRAPEFRUIT | 475.05 | 0.000037580 |
| ETHYL LINALOOL | 5.7000 | CITRUS | FLORAL | 275.53 | 0.000015198 |
| ROSSITOL | 3.0000 | NUGUET | CITRUS | 303.80 | 0.000020753 |
| CIS 3 HEXENOL | 0.0500 | GRASS | GREEN | 1569.11 | 0.000183540 |
| METHYL OCTINE CARBONATE | 0.4000 | GREEN | VIOLET | 525.10 | 0.000036464 |
| Water Release Group 4 | | | | | |
| TRANS 2 CIS 6 NONADENOL | 0.4500 | GREEN | CUCUMBER | 245.87 | 0.000012085 |
| GARDAMIDE | 5.0000 | CITRUS | WOODY | 86.47 | 0.000002154 |
| UNDECAVERTOL | 1.5000 | GREEN | FRUITY | 118.38 | 0.000004491 |
| ALDEHYDE C12 (DODECANAL) | 0.4000 | ALDEHYDIC | FATTY/GREASY | 183.88 | 0.000008217 |
| Water Release Group 5 | | | | | |
| CIS 4 DECENAL | 0.0043 | ALDEHYDIC | CARDAMOM | 1078.65 | 0.000090457 |
| TRANS 2 CIS 6 NONADENAL | 0.0040 | GREEN | CUCUMBER | 1010.87 | 0.000075353 |
| MEFRANAL | 0.4000 | ALDEHYDIC | CITRUS | 87.76 | 0.000003486 |
| CITRATHAL | 1.5000 | LIME | CITRUS | 39.32 | 0.000001915 |
| Water Release Group 6 | | | | | |
| PARADISAMIDE | 7.0000 | CITRUS | FRUITY | 7.77 | 0.000000242 |
| TRIDECEN 2 NITRILE | 0.5000 | NITRILE | CITRUS | 6.43 | 0.000000137 |
| CIS-3-HEXENYL SALICYLATE | 0.5000 | GRASS | SALICYLATE | 2.30 | 0.000000069 |
| Perfume Total | 100.0033 | | | | |

| | | | % Odor |
|---|---|---|---|
| | Ω | odt | Contribution |
| Water Release Group 1 | | | |
| d-LIMONINE | 544.607548652 | 430 | 100.00 |
| | | % Citrus | 100.00 |
| | | % Green | 0.00 |
| Water Release Group 2 | | | |
| STRALLYL ACETATE | 1.483977879 | 110 | 1.54 |
| DIHYDRO MYRCENOL | 0.952972906 | 810 | 2.22 |
| MELONAL | 0.411493829 | 1.5 | 40.04 |
| LIGUSTRAL | 0.257251791 | 110 | 0.84 |
| CITRONELLAL | 0.251072029 | 33 | 5.46 |
| CITRONELLYL NITRILE | 0.215951073 | 71 | 7.81 |
| CIS 3 HEXENYL ACETATE | 0.090121564 | 170 | 0.36 |
| LINALOOL | 0.076925400 | 20 | 15.02 |
| ALDEHYDE C 8 (OCTANAL) | 0.074341612 | 4.8 | 1.18 |
| ALDEHYDE C 10 (DECANAL) | 0.070875843 | 8.7 | 25.84 |
| | | % Citrus | 42.11 |
| | | % Green | 42.52 |
| Water Release Group 3 | | | |
| RHUBAFURAN | 0.053871058 | 440 | 8.82 |
| ETHYL LINALOOL | 0.023577881 | 120 | 48.16 |
| ROSSITOL | 0.018914598 | 440 | 8.82 |
| CIS 3 HEXENOL | 0.012830600 | 90 | 0.54 |
| METHYL OCTINE CARBONATE | 0.007658962 | 9.7 | 40.08 |
| | | % Citrus | 52.77 |
| | | % Green | 47.23 |
| Water Release Group 4 | | | |
| TRANS 2 CIS 6 NONADENOL | 0.001327108 | 1.5 | 50.89 |
| GARDAMIDE | 0.001051879 | 24 | 35.34 |
| UNDECAVERTOL | 0.000784043 | 28 | 9.79 |
| ALDEHYDE C12 (DODECANAL) | 0.000677932 | 17 | 3.99 |
| | | % Citrus | 35.34 |
| | | % Green | 60.67 |
| Water Release Group 5 | | | |
| CIS 4 DECENAL | 0.000418780 | 3.1 | 0.42 |
| TRANS 2 CIS 6 NONADENAL | 0.000304629 | 0.2 | 8.10 |
| MEFRANAL | 0.000118183 | 17 | 7.16 |
| CITRATHAL | 0.000115109 | 5.3 | 88.30 |
| | | % Citrus | 93.48 |
| | | % Green | 8.10 |
| Water Release Group 6 | | | |
| PARADISAMIDE | 0.000013163 | 0.6 | 95.75 |
| TRIDECEN 2 NITRILE | 0.000000441 | 8.7 | 0.52 |
| CIS-3-HEXENYL SALICYLATE | 0.000000150 | 1.9 | 2.82 |
| | | % Citrus | 97.38 |
| | | % Green | 2.28 |
| Perfume Total | | | |

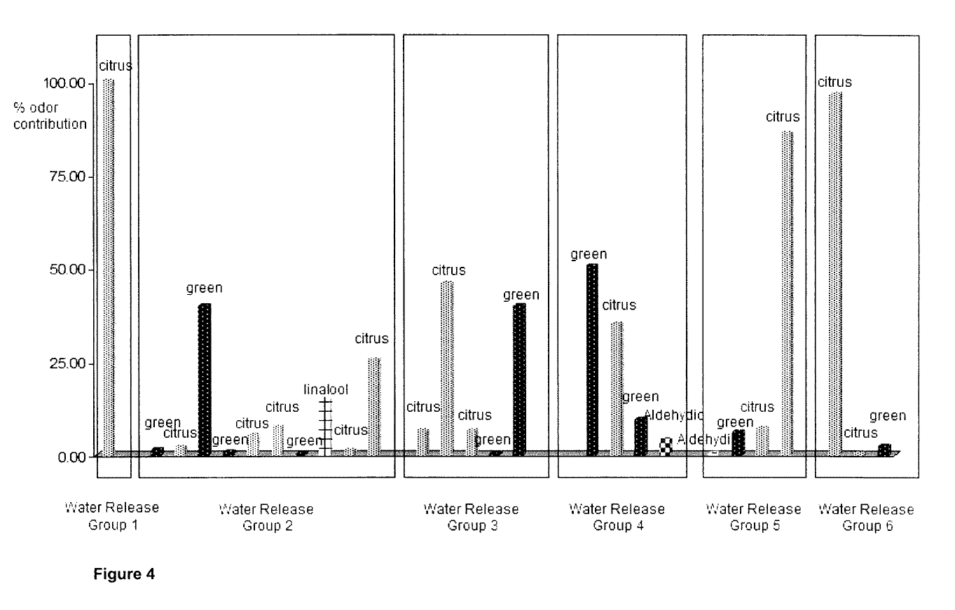

The odor profile of each odorant in each water release group is expressed in percentage contribution according to odor type. This kinetic odor progression of the perfume in rinse-off conditions based on each odorant's odor contribution is shown in FIG. 4.

The flash release odorants (γ values more than 900) and the deposition odorants (also referred to as delayed release odorants: γ values less than 100) are calculated to make up respectively 52% and 15% of the total perfume. The acceleration (γ) values of the fragrance odorants are shown in table 16:

TABLE 16

| | Parts | γ |
|---|---|---|
| Flash Release Materials | | |
| d-LIMONENE | 42.2500 | 8200.76 |
| ALDEHYDE C 8 (OCTANAL) | 0.0450 | 3630.08 |
| MELONAL | 0.5000 | 2655.52 |
| STYRALLYL ACETATE | 1.5000 | 2576.59 |
| LIGUSTRAL | 0.7700 | 1704.65 |
| CIS 3 HEXENOL | 0.0500 | 1569.11 |
| CIS 3 HEXENYL ACETATE | 0.5000 | 1384.27 |
| CITRONELLAL | 1.5000 | 1345.09 |
| CIS 4 DECENAL | 0.0043 | 1076.65 |
| TRANS 2 CIS 6 NONADIENAL | 0.0040 | 1010.67 |
| CITRONELLYL NITRILE | 4.5000 | 913.04 |
| Total | 51.6233 | |
| Sustained Release Materials | | |
| DIHYDRO MYRCENOL | 15.0000 | 866.55 |
| ALDEHYDE C10 (DECANAL) | 1.4300 | 816.26 |
| LINALOOL | 2.5000 | 644.41 |
| METHYL OCTINE CARBONATE | 0.4000 | 525.10 |
| RHUBAFURAN | 3.0000 | 476.08 |
| ROSSITOL | 3.0000 | 303.80 |
| ETHYL LINALOOL | 5.7000 | 275.63 |
| TRANS 2 CIS 6 NONADIENOL | 0.4500 | 245.87 |
| ALDEHYDE C12 (DODECANAL) | 0.4000 | 183.88 |
| UNDECAVERTOL | 1.5000 | 116.38 |
| Total | 33.3800 | |
| Delayed Release Materials | | |
| MEFRANAL | 0.4000 | 84.76 |
| GARDAMIDE | 5.0000 | 66.47 |
| CITRATHAL | 1.5000 | 39.32 |
| PARADISAMIDE | 7.0000 | 7.77 |
| TRIDECEN 2 NITRILE | 0.5000 | 6.43 |
| CIS-3-HEXENYL SALICYLATE | 0.6000 | 2.80 |
| Total | 15.0000 | |

The above perfume example provides a citrus linear hedonic with a secondary linear cucumber odor while also leaving a minimum amount of residual fragrance of streaks upon completing the cycle or cleaning experience.

### B. Body-Wash, Soap, Shampoo and Conditioners

Below in Table 17 is an illustrative example (“Citrus Cucumber Sustained Release-Type”) of a fragrance engineered for sustained release of a dominating citrus with a secondary linear green cucumber note in high water dilutions.

As in the earlier example for “Flash Release Citrus-Cucumber”, a linear citrus note can be constructed by ensuring that least three, preferably four of the water release groups constructed based on odorants' Ω values must have at least 30%, preferably at least 40% of their overall odor contributed by one or more citrus odorants. In addition a linear release of a secondary green nuance along with the predominant citrus release may be built by having at least three, preferably four of the water release groups to have at least 20%, of their overall odor contributed by one or more green odorants

The Citrus-Cucumber Sustained Release perfume is designed based on the same criteria defined earlier for sustained release in water: at least 30%, preferably at least 40% of the total perfume with γ values between 900 and 100. The perfume is shown in the table below:

TABLE 17
Citrus-Cucumber Sustained Release Type Fragrance

| | | Odor | Odor | |
|---|---|---|---|---|
| | Parts | Descriptor 1 | Descriptor 2 | γ |
| Water Release Group 1 | | | | |
| d-LIMONINE | 10.0000 | CITRUS | ORANGE | 8200.76 |
| Water Release Group 2 | | | | |
| STRALLYL ACETATE | 1.5000 | GREEN | FRUITY | 2576.59 |
| DIHYDRO MYRCENOL | 15.0000 | CITRUS | METALLIC | 888.55 |
| MELONAL | 0.5000 | GREEN | ALDEHYDIC | 2555.52 |
| LIGUSTRAL | 0.7700 | GREEN | LEAF | 1704.65 |
| CITRONELLAL | 1.5000 | CITRUS | CITRONELLA | 1345.08 |
| CITRONELLYL NITRILE | 4.5000 | CITRUS | NITRILE | 913.04 |
| LINALOOL | 0.5000 | LINALOOL | | 844.41 |
| ALDEHYDE C 10 (DECANAL) | 2.3600 | ALDEHYDIC | ORANGE | 615.25 |
| CIS 3 HEXENYL ACETATE | 0.5000 | GRASS | FRUITY | 1384.27 |
| RHUBAFURAN | 5.0000 | GREEN | GRAPEFRUIT | 478.05 |
| ALDEHYDE C 8 (OCTANAL) | 0.0450 | ALDEHYDIC | CITRUS | 3830.08 |
| Water Release Group 3 | | | | |
| ROSSITOL | 15.0000 | CITRUS | FLORAL | 276.63 |
| ETHYL LINALOOL | 0.5000 | MUGUET | CITRUS | 303.86 |
| CIS 3 HEXENOL | 0.0500 | GRASS | GREEN | 1569.11 |
| METHYL OCTINE CARBONATE | 0.4000 | GREEN | VIOLET | 525.10 |
| Water Release Group 4 | | | | |
| GARDAMIDE | 10.0000 | CITRUS | WOODY | 86.47 |
| UNDECAVERTOL | 3.5000 | GREEN | FRUITY | 118.38 |
| TRANS 2 CIS 6 NONADENOL | 0.4500 | GREEN | CUCUMBER | 245.87 |
| ALDEHYDE C12 (DODECANAL) | 0.4000 | ALDEHYDIC | FATTY/GREASY | 183.88 |
| Water Release Group 5 | | | | |
| CITRATHAL | 5.5000 | LIME | CITRUS | 29.32 |
| CIS 4 DECENAL | 0.0043 | ALDEHYDIC | CARDAMOM | 1075.68 |
| TRANS 2 CIS 6 NONADENAL | 0.0040 | GREEN | CUCUMBER | 1010.67 |
| Water Release Group 6 | | | | |
| MEFRANAL | 0.4000 | ALDEHYDIC | CITRUS | 94.78 |
| PARADISAMIDE | 3.0000 | CITRUS | FRUITY | 7.77 |
| TRIDECEN 2 NITRILE | 0.5000 | NITRILE | CITRUS | 8.43 |
| CIS-3-HEXENYL SALICYLATE | 0.6000 | GRASS | SALICYLATE | 2.80 |
| Perfume Total | 100.0033 | | | |

| | | | | % Odor |
|---|---|---|---|---|
| | φ | Ω | odt | Contribution |
| Water Release Group 1 | | | | |
| d-LIMONINE | 0.001571820 | 128.901194947 | 430 | 100.00 |
| | | | % Citrus | 100.00 |
| | | | % Green | 0.00 |
| Water Release Group 2 | | | | |
| STRALLYL ACETATE | 0.000383964 | 1.483977879 | 110 | 1.23 |
| DIHYDRO MYRCENOL | 0.000073216 | 0.952972908 | 810 | 1.87 |
| MELONAL | 0.000309918 | 0.411493829 | 1.5 | 30.01 |
| LIGUSTRAL | 0.000196597 | 0.257251791 | 110 | 0.63 |
| CITRONELLAL | 0.000124439 | 0.251072020 | 33 | 4.09 |
| CITRONELLYL NITRILE | 0.000053290 | 0.218951073 | 71 | 5.71 |
| LINALOOL | 0.000047748 | 0.153850800 | 20 | 22.51 |
| ALDEHYDE C 10 (DECANAL) | 0.000060573 | 0.117962667 | 6.7 | 31.97 |
| CIS 3 HEXENYL ACETATE | 0.000130208 | 0.696121584 | 170 | 0.28 |
| RHUBAFURAN | 0.000037530 | 0.089451785 | 440 | 1.02 |
| ALDEHYDE C 8 (OCTANAL) | 0.000456095 | 0.074341812 | 4.5 | 0.85 |
| | | | % Citrus | 44.33 |
| | | | % Green | 33.16 |
| Water Release Group 3 | | | | |
| ROSSITOL | 0.000015198 | 0.062636476 | 120 | 87.18 |
| ETHYL LINALOOL | 0.000020753 | 0.053591356 | 440 | 10.38 |
| CIS 3 HEXENOL | 0.000183540 | 0.012830800 | 90 | 0.30 |
| METHYL OCTINE CARBONATE | 0.000035454 | 0.007858962 | 8.7 | 22.18 |
| | | | % Citrus | 77.64 |
| | | | % Green | 22.46 |
| Water Release Group 4 | | | | |
| GARDAMIDE | 0.000003164 | 0.002103357 | 24 | 47.83 |
| UNDECAVERTOL | 0.000004491 | 0.001829435 | 28 | 15.39 |
| TRANS 2 CIS 6 NONADENOL | 0.000012085 | 0.001337108 | 1.5 | 34.28 |
| ALDEHYDE C12 (DODECANAL) | 0.000009217 | 0.000577933 | 17 | 2.69 |
| | | | % Citrus | 47.63 |
| | | | % Green | 49.88 |
| Water Release Group 5 | | | | |
| CITRATHAL | 0.000001915 | 0.000422065 | 5.3 | 97.98 |
| CIS 4 DECENAL | 0.000000457 | 0.000418780 | 3.1 | 0.13 |
| TRANS 2 CIS 6 NONADENAL | 0.000075353 | 0.000304829 | 0.2 | 1.89 |
| | | | % Citrus | 97.98 |
| | | | % Green | 2.02 |
| Water Release Group 6 | | | | |
| MEFRANAL | 0.000003488 | 0.000013133 | 17 | 0.17 |
| PARADISAMIDE | 0.000000242 | 0.000015044 | 0.6 | 98.98 |
| TRIDECEN 2 NITRILE | 0.000000137 | 0.000000441 | 6.7 | 0.54 |
| CIS-3-HEXENYL SALICYLATE | 0.000000069 | 0.000000150 | 1.9 | 2.30 |
| | | | % Citrus | 97.70 |
| Perfume Total | | | % Green | 2.30 |

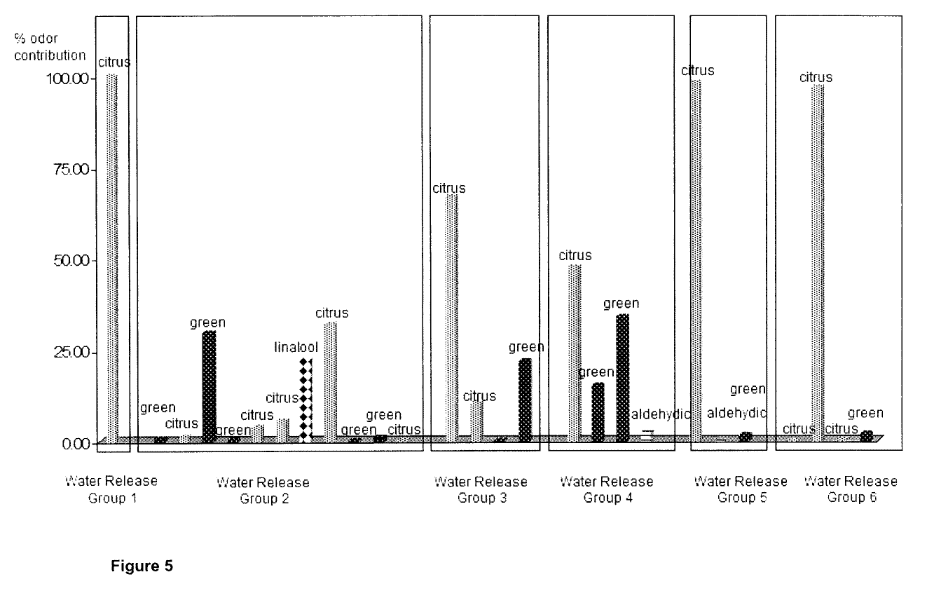

The odor profile of each odorant in each water release group is expressed in percentage contribution according to odor type. This kinetic odor progression of the perfume in rinse-off conditions based on each odorant's odor contribution is shown in FIG. 5.

Odorants from the “Citrus-Cucumber Linear Sustained Release” type are also grouped according to their type of release based on the γ values as shown below in table 18:

| | Parts | γ |
|---|---|---|
| Flash Release Odorants | | |
| d-LIMONENE | 10.0000 | 8200.76 |
| ALDEHYDE C 8 (OCTANAL) | 0.0450 | 3630.08 |
| MELONAL | 0.5000 | 2655.52 |
| STYRALLYL ACETATE | 1.5000 | 2578.59 |
| LIGUSTRAL | 0.7700 | 1704.65 |
| CIS 3 HEXENOL | 0.0500 | 1569.11 |
| CIS 3 HEXENYL ACETATE | 0.5000 | 1384.27 |
| CITRONELLAL | 1.5000 | 1345.09 |
| CIS 4 DECENAL | 0.0043 | 1076.85 |
| TRANS 2 CIS 6 NONADIENAL | 0.0040 | 1010.67 |
| CITRONELLYL NITRILE | 4.5000 | 913.04 |
| | 19.3733 | |
| Sustained Release Odorants | | |
| DIHYDRO MYRCENOL | 15.0000 | 866.55 |
| ALDEHYDE C10 (DECANAL) | 2.3800 | 818.26 |
| LINALOOL | 5.0000 | 644.41 |
| METHYL OCTINE CARBONATE | 0.4000 | 525.10 |
| RHUBAFURAN | 5.0000 | 476.06 |
| ROSSITOL | 8.5000 | 303.80 |
| ETHYL LINALOOL | 15.0000 | 275.63 |
| TRANS 2 CIS 6 NONADIENOL | 0.4500 | 245.87 |
| ALDEHYDE C12 (DODECANAL) | 0.4000 | 183.88 |
| UNDECAVERTOL | 3.5000 | 116.38 |
| | 55.6300 | |
| Delayed Release Odorants | | |
| MEFRANAL | 0.4000 | 84.76 |
| GARDAMIDE | 10.0000 | 66.47 |
| CITRATHAL | 5.5000 | 39.32 |
| PARADISAMIDE | 8.0000 | 7.77 |
| TRIDECEN 2 NITRILE | 0.5000 | 6.43 |
| CIS-3-HEXENYL SALICYLATE | 0.6000 | 2.80 |
| | 25.0000 | |

The above perfume example provides a linear sustained citrus dominating odor with a secondary cucumber release during rinse-off.

### C. Laundry Products

The following example is illustrative of a linear dominating citrus release with a secondary linear cucumber hedonic note for leave-on applications. Perfumes intended for maximum deposition in wash-off systems must have at least 40% and preferably at least 50% of the total perfume with delayed release type of odorants (depositors) as defined in the invention.

In addition to criteria for maximum deposition of perfume defined above, at least three, preferably four of the water release groups constructed based on odorants' Ω values must have at least 30%, preferably at least 40% of their overall odor contributed by one or more citrus odorants. In addition, to construct a secondary linear green note, at least three water release groups based on Ω values, must have at least 20% of their overall odor contributed by a single or a group of green odorants. These fragrances will therefore also provide the consumer with a perception of linear sustained predominantly citrus perfume with a linear nuance of cucumber throughout the process of rinse-off.

A perfume (Delayed Linear Release Cucumber-Citrus Perfume) for laundry detergents designed to provide maximum deposition of fragrance as well as a linear release of a citrus/green note during the process of rinse-off is shown below in Table 19.

TABLE 19

| | | Odor | Odor | | | | | % Odor |
|---|---|---|---|---|---|---|---|---|
| | Parts | Descriptor 1 | Descriptor 2 | γ | φ | Ω | odt | Contribution |
| Water Release Group 1 | | | | | | | | |
| d-LIMONINE | 2.5 | CITRUS | ORANGE | 8200.76 | 0.0015718 | 32.2252987 | 430 | 100.00 |
| | | | | | | | % Citrus | 100.00 |
| | | | | | | | % Green | 0.00 |
| Water Release Group 2 | | | | | | | | |
| STRALLYL ACETATE | 1.5 | GREEN | FRUITY | 2576.58 | 0.0003840 | 1.4838779 | 110 | 1.42 |
| MELONAL | 0.5 | GREEN | ALDEHYDIC | 2655.52 | 0.0003099 | 0.4114938 | 1.5 | 34.76 |
| LIGUSTRAL | 0.77 | GREEN | LEAF | 1704.85 | 0.0001950 | 0.2572618 | 110 | 0.73 |
| CITRONELLAL | 1.5 | CITRUS | CITRONELLA | 1345.09 | 0.0001244 | 0.2510720 | 33 | 4.74 |
| CITRONELLYL NITRILE | 4.5 | CITRUS | NITRILE | 913.04 | 0.0000523 | 0.2189511 | 71 | 5.61 |
| DIHYDRO MYRCENOL | 2.5 | CITRUS | METALLIC | 868.55 | 0.0000733 | 0.1588288 | 810 | 0.32 |
| ALDEHYDE C10 (DECANAL) | 2.38 | ALDEHYDIC | CITRUS | 818.26 | 0.0000808 | 0.1179829 | 6.7 | 37.05 |
| CIS 3 HEXENYL ACETATE | 0.5 | GRASS | FRUITY | 1384.27 | 0.0001302 | 0.0901218 | 170 | 0.31 |
| LINALOOL | 2.5 | LINALOOL | | 544.41 | 0.0000477 | 0.0759254 | 20 | 13.04 |
| ALDEHYDE C 8 (OCTANAL) | 0.045 | ALDEHYDIC | CITRUS | 3630.08 | 0.0004551 | 0.0743418 | 4.8 | 1.02 |
| | | | | | | | % Citrus | 49.74 |
| | | | | | | | % Green | 37.22 |
| Water Release Group 3 | | | | | | | | |
| ETHYL LINALOOL | 15 | CITRUS | FLORAL | 276.63 | 0.0000152 | 0.0528356 | 120 | 65.96 |
| ROSSITOL | 8.5 | MUGUET | CITRUS | 303.86 | 0.0000206 | 0.0535914 | 440 | 10.18 |
| RHUBAFURAN | 1.6 | GREEN | GRAPEFRUIT | 475.08 | 0.0000375 | 0.0258365 | 440 | 1.80 |
| CIS 3 HEXENOL | 0.05 | GRASS | GREEN | 1569.11 | 0.0001835 | 0.0128308 | 90 | 0.29 |
| METHYL OCTINE CARBONATE | 0.4 | GREEN | VIOLET | 525.10 | 0.0000356 | 0.0075690 | 9.7 | 21.75 |
| | | | | | | | % Citrus | 76.15 |
| | | | | | | | % Green | 23.85 |
| Water Release Group 4 | | | | | | | | |
| MEFRANAL | 12 | ALDEHYDIC | CITRUS | 84.76 | 0.0000035 | 0.0035455 | 17 | 39.46 |
| GARDAMIDE | 15 | CITRUS | WOODY | 56.47 | 0.0000032 | 0.0031550 | 24 | 34.94 |
| UNDECAVERTOL | 3.5 | GREEN | FRUITY | 116.38 | 0.0000045 | 0.0012294 | 28 | 7.52 |
| TRANS 2 CIS 6 NONADENOL | 0.45 | GREEN | CUCUMBER | 245.57 | 0.0000121 | 0.0013371 | 1.5 | 16.77 |
| ALDEHYDE C12 (DODECANAL) | 0.4 | ALDEHYDIC | FATTY/GREASY | 183.88 | 0.0000092 | 0.0005779 | 17 | 1.32 |
| | | | | | | | % Citrus | 74.39 |
| | | | | | | | % Green | 24.29 |
| Water Release Group 5 | | | | | | | | |
| CIS 4 DECENAL | 0.0043 | ALDEHYDIC | CARDAMOM | 1078.65 | 0.0000905 | 0.0004186 | 3.1 | 0.28 |
| TRANS 2 CIS 6 NONADENAL | 0.004 | GREEN | CUCUMBER | 1010.87 | 0.0000754 | 0.0003046 | 0.2 | 4.08 |
| CITRATHAL | 2.5 | LIME | CITRUS | 39.32 | 0.0000019 | 0.0001918 | 5.3 | 95.68 |
| | | | | | | | % Citrus | 95.68 |
| | | | | | | | % Green | 4.06 |
| Water Release Group 6 | | | | | | | | |
| PARADISAMIDE | 8 | CITRUS | FRUITY | 7.77 | 0.0000002 | 0.0000156 | 0.8 | 33.85 |
| METHYL DIHYDRO JASMONATE | 5 | FLORAL | | 8.40 | 0.0000003 | 0.0000120 | 0.23 | 55.23 |
| CIS-3-HEXENYL SALICYLATE | 8 | GRASS | SALICYLATE | 2.80 | 0.0000001 | 0.0000020 | 1.9 | 10.70 |
| TRIDECEN 2 NITRILE | 0.5 | NITRILE | CITRUS | 6.43 | 0.0000001 | 0.0000004 | 8.7 | 0.18 |
| | | | | | | | % Citrus | 34.07 |
| | | | | | | | % Green | 10.70 |

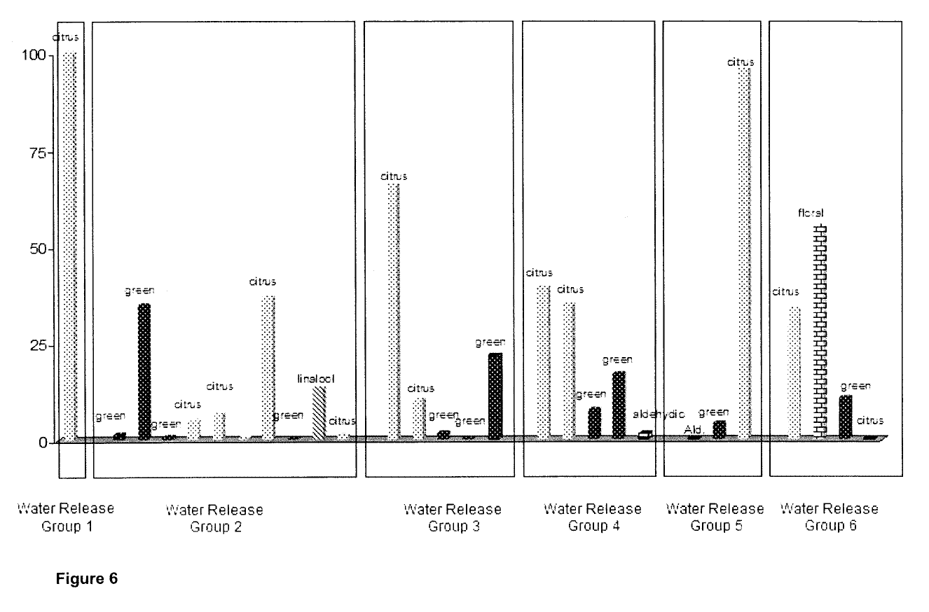

The odor profile of each odorant in each water release group is expressed in percentage contribution according to odor type. This kinetic odor progression of the perfume in rinse-off conditions based on each odorant's odor contribution is shown in FIG. 6.

The odorants in the illustrative example are also grouped according to their type of release based on the acceleration (γ) values as shown in table 20 below.

TABLE 20

| | Parts | γ |
|---|---|---|
| Flash Release | | |
| d-LIMONENE | 2.5000 | 8200.76 |
| ALDEHYDE C 8 (OCTANAL) | 0.0450 | 3630.08 |
| MELONAL | 0.5000 | 2655.52 |
| STYRALLYL ACETATE | 1.5000 | 2576.59 |
| LIGUSTRAL | 0.7700 | 1704.65 |
| CIS 3 HEXENOL | 0.0500 | 1569.11 |
| CIS 3 HEXENYL ACETATE | 0.5000 | 1384.27 |
| CITRONELLAL | 1.5000 | 1345.09 |
| CIS 4 DECENAL | 0.0043 | 1078.65 |
| TRANS 2 CIS 6 NONADIENAL | 0.0040 | 1010.67 |
| CITRONELLYL NITRILE | 4.5000 | 913.04 |
| total | 11.8733 | |
| Sustained Release | | |
| DIHYDRO MYRCENOL | 2.5000 | 866.55 |
| ALDEHYDE C10 (DECANAL) | 2.3800 | 818.26 |
| LINALOOL | 2.5000 | 644.41 |
| METHYL OCTINE CARBONATE | 0.4000 | 525.10 |
| RHUBAFURAN | 1.5000 | 476.06 |
| ROSSITOL | 8.5000 | 303.80 |
| ETHYL LINALOOL | 15.0000 | 275.63 |
| TRANS 2 CIS 6 NONADIENOL | 0.4500 | 245.87 |
| ALDEHYDE C12 (DODECANAL) | 0.4000 | 183.88 |
| UNDECAVERTOL | 3.5000 | 116.36 |
| total | 37.1300 | |
| Delayed Release | | |
| MEFRANAL | 12.0000 | 84.76 |
| GARDAMIDE | 15.0000 | 66.47 |
| CITRATHAL | 2.5000 | 39.32 |
| METHYL DIHYDRO JASMPNATE | 5.0000 | 6.40 |
| PARADISAMIDE | 8.0000 | 7.77 |
| TRIDECEN 2 NITRILE | 0.5000 | 6.43 |
| CIS-3-HEXENYL SALICYLATE | 8.0000 | 2.80 |
| total | 51.0000 | |

“Delayed Linear Release Cucumber-Citrus Perfume” for laundry detergents provides maximum deposition of fragrance as well as a linear release of a citrus/green note during the process of rinse-off in use.

In addition to the Citrus-Green examples provided above, examples below will in turn, provide illustrations of perfumes for linear citrus release with linear nuances of floral and fruity odors in rinse-off.

The method to construct these Citrus-Fruity and Citrus-Floral perfumes is the same as the ones shown for Citrus-Green.

### Citrus-Fruity Perfumes

All the following perfumes will result in a predominantly linear citrus odor during rinse off whilst also providing the consumer with a linear perception of a secondary fruity nuance.

### A. Flash Release Citrus-Fruity

The following provided example “Flash Release Citrus Fruity” perfume is for applications intended to result in minimal deposition of fragrance upon rinse-off such as dishwashing liquid and glass cleaners. The example of Flash Release Citrus Fruity is shown below in table 21.

TABLE 21

| | Parts | γ | φ | Ω | ODT | % Odor |
|---|---|---|---|---|---|---|
| Water Release Group 1 | | | | | | |
| d-LIMONINE | 35 | 8200.7592 | 0.0015718 | 451.15418 | 430 | 76.50 |
| ETHYL 2-METHYLBUTYRATE | 0.5 | 12827.563 | 0.0043615 | 27.973938 | 20 | 23.50 |
| | | | | | % Citrus | 76.50 |
| | | | | | % Fruity | 23.50 |
| Water Release Group 2 | | | | | | |
| MANZANATE | 0.5 | 5288.4237 | 0.00111863 | 2.9518233 | 50 | 0.76 |
| CITRONELLAL | 7.5 | 1345.0902 | 0.0001244 | 1.2553601 | 33 | 17.16 |
| DIHYDROMYRCENOL | 10 | 888.54502 | 7.332E−05 | 0.6353153 | 810 | 0.93 |
| ALDEHYDE C 8 (OCTANAL) | 0.3 | 3830.0763 | 0.0004551 | 0.4956107 | 290 | 0.06 |
| CITRONELLYL NITRILE | 6 | 913.04218 | 5.329E−05 | 0.2919348 | 71 | 6.39 |
| ORTHOLATE | 9.5 | 584.56181 | 6.262E−05 | 0.2828299 | 58 | 12.38 |
| CITRAL | 5 | 857.09011 | 6.236E−05 | 0.2672318 | 12 | 31.50 |
| ALLYL CAPROATE | 0.5 | 1738.6656 | 0.0002108 | 0.183027 | 4.6 | 8.22 |
| ALDEHYDE C10 | 2 | 818.26196 | 8.057E−05 | 0.0991285 | 6.7 | 22.57 |
| | | | | | % Citrus | 78.65 |
| | | | | | % Fruity | 21.35 |
| Water Release Group 3 | | | | | | |
| LINALYL ACETATE | 1.5 | 617.71622 | 8.451E−05 | 0.059773 | 450 | 2.07 |
| APPLINAL | 1.5 | 554.78818 | 8.246E−05 | 0.0519759 | 55 | 16.97 |
| HEXYL ACETATE | 0.8 | 3118.7849 | 0.0004649 | 0.0464022 | 950 | 0.52 |
| RHUBAFURAN | 2 | 478.05986 | 3.758E−05 | 0.0357807 | 440 | 2.83 |
| ALLYL HEPTANOATE | 0.5 | 711.57994 | 8.076E−05 | 0.0216168 | 58 | 5.36 |
| APHERMATE | 0.5 | 589.61679 | 5.808E−05 | 0.0165332 | 100 | 3.11 |
| DIMETHYL BENZYL CARBINYL ACETATE | 2 | 249.9309 | 1.784E−05 | 0.0089185 | 18 | 89.13 |
| | | | | | % Citrus | 4.90 |
| | | | | | % Fruity | 95.10 |
| Water Release Group 4 | | | | | | |
| OXANE | 0.1 | 294.09802 | 3.802E−05 | 0.0011181 | 58 | 4.82 |
| CIS 4 DECENAL | 0.008 | 1076.6476 | 9.046E−05 | 0.0005843 | 3.1 | 5.22 |
| PHENOXY ETHYL ISOBUTYRATE | 4 | 52.666397 | 2.538E−05 | 0.0005346 | 120 | 89.98 |
| | | | | | % Citrus | 10.04 |
| | | | | | % Fruity | 89.96 |
| Water Release Group 5 | | | | | | |
| GRAPEFRUIT MERCAPTAN | 0.003 | 1043.221 | 0.0001031 | 0.0003227 | 0.00002 | 98.99 |
| CITRATHAL | 4 | 39.32 | 1.915E−06 | 0.000307 | 5.3 | 0.50 |
| alpha-DAMASCONE | 0.1 | 157.3017 | 9.183E−06 | 0.0001444 | 3.6 | 0.02 |
| DIMETHYL BENZYL CARBINYL BUTYRATE | 1.5 | 39.667071 | 1.85E−06 | 0.0001106 | 42 | 0.02 |
| GAMMA UNDECALACTONE | 0.5 | 42.962736 | 1.513E−06 | 3.251E−05 | 0.7 | 0.47 |
| | | | | | % Citrus | 99.49 |
| | | | | | % Fruity | 0.51 |
| Water Release Group 6 | | | | | | |
| ISO E SUPER | 0.75 | 27.835581 | 1.397E−06 | 2.915E−05 | 0.6 | 16.27 |
| NECTARYL | 1.55 | 13.535512 | 5.134E−07 | 1.077E−05 | 0.4 | 50.42 |
| PARADISAMIDE | 1.5 | 7.7363238 | 2.42E−07 | 2.821E−06 | 0.6 | 32.53 |
| TRIDECEN 2 NITRILE | 0.4 | 6.426612 | 1.372E−07 | 3.527E−07 | 6.7 | 0.78 |
| | | | | | % Citrus | 83.73 |
| | | | | | % Fruity | 0.00 |

The above perfume results in an impactful citrus linear note during dilution coupled with linear nuances of apple throughout usage.

### B. Sustained Release Citrus-Fruity Perfume

The following perfume “Sustained Release Citrus-Fruity Perfume” is an example of a perfume resulting in a sustained linear predominantly citrus note with clear linear nuances of fruit in high water dilutions. This perfume is intended for applications such as shampoo, conditioners, soap etc. and is designed based on methods discussed in great details earlier in the herein invention.

The perfume “Sustained Release Citrus-Fruity” analysis along with its composition is shown below in table 22:

| | Parts | γ | φ | Ω | ODT | % Odor |
|---|---|---|---|---|---|---|
| Release Group I | | | | | | |
| d-LIMONINE | 15.2500 | 8200.76 | 0.001571820 | 195.574322295 | 430 | 73.94 |
| ETHYL 2-METHYLBUTYRATE | 0.2500 | 12827.56 | 0.004361536 | 13.986969219 | 20 | 26.06 |
| | | | | | % Citrus | 73.94 |
| | | | | | % Fruity | 26.06 |
| Release Group II | | | | | | |
| MANZANATE | 0.5000 | 5288.42 | 0.001116334 | 2.951823345 | 50 | 0.70 |
| CITRONELLAL | 10.0000 | 1345.09 | 0.000124439 | 1.673813464 | 33 | 21.25 |
| DIHYDROMYRCENOL | 15.0000 | 866.55 | 0.000073316 | 0.952972906 | 810 | 1.30 |
| ALDEHYDE C 8 (OCTANAL) | 0.3000 | 3630.08 | 0.000455096 | 0.495810745 | 290 | 0.07 |
| CITRONELLYL NITRILE | 6.0000 | 913.04 | 0.000053290 | 0.291934763 | 71 | 5.93 |
| ORTHOLATE | 9.5000 | 564.56 | 0.000062622 | 0.282829871 | 58 | 11.49 |
| CITRAL | 5.0000 | 857.09 | 0.000062358 | 0.267231617 | 12 | 29.22 |
| ALLYL CAPROATE | 0.5000 | 1738.67 | 0.000217080 | 0.183027006 | 4.8 | 7.52 |
| LINALYL ACETATE | 4.5000 | 617.72 | 0.000054510 | 0.179319015 | 450 | 0.70 |
| ALDEHYDE C10 | 2.0000 | 818.28 | 0.000060573 | 0.099128451 | 6.7 | 20.93 |
| RHUBAFURAN | 5.0000 | 478.08 | 0.000037580 | 0.089451765 | 440 | 0.50 |
| | | | | | % Citrus | 80.19 |
| | | | | | % Fruity | 19.81 |
| Release Group III | | | | | | |
| APPLINAL | 1.5000 | 554.79 | 0.000062457 | 0.051975865 | 55 | 16.75 |
| APHERMATE | 1.5000 | 589.82 | 0.000058081 | 0.049599731 | 100 | 9.21 |
| HEXYL ACETATE | 0.5000 | 3118.78 | 0.000464946 | 0.045402167 | 950 | 0.52 |
| ALLYL HEPTANOATE | 0.5000 | 711.58 | 0.000060757 | 0.021616782 | 58 | 5.29 |
| DIMETHYL BENZYL CARBINYL ACETATE | 2.0000 | 249.93 | 0.000017842 | 0.008918540 | 18 | 88.23 |
| | | | | | % Citrus | 0.00 |
| | | | | | % Fruity | 100.00 |
| Release Group IV | | | | | | |
| OXANE | 0.5000 | 294.10 | 0.000038018 | 0.005590490 | 56 | 4.88 |
| alpha-DAMASCONE | 0.5000 | 157.30 | 0.000009183 | 0.000722246 | 3.6 | 75.86 |
| CIS 4 DECENAL | 0.0080 | 1078.85 | 0.000090457 | 0.000584344 | 3.1 | 1.06 |
| PHENOXY ETHYL ISOBUTYRATE | 4.0000 | 52.67 | 0.000002538 | 0.000534648 | 120 | 18.21 |
| | | | | | % Citrus | 5.93 |
| | | | | | % Fruity | 94.07 |
| Water Release Group V | | | | | | |
| GRAPEFRUIT MERCAPTAN | 0.0030 | 1043.22 | 0.000103096 | 0.000322655 | 0.00002 | 99.01 |
| CITRATHAL | 4.0000 | 39.32 | 0.000001915 | 0.000308956 | 5.3 | 0.50 |
| DIMETHYL BENZYL CARBINYL BUTYRATE | 1.5000 | 39.87 | 0.000001850 | 0.000110829 | 42 | 0.02 |
| GAMMA UNDECALACTONE | 0.5000 | 4.98 | 0.000001513 | 0.000032513 | 0.7 | 0.47 |
| | | | | | % Citrus | 99.50 |
| | | | | | % Fruity | 0.50 |
| Release Group VI | | | | | | |
| NECTARYL | 3.0000 | 13.54 | 0.000000513 | 0.000020848 | 0.4 | 44.84 |
| ISO E SUPER | 0.5000 | 27.84 | 0.000001397 | 0.000019436 | 0.6 | 4.98 |
| PARADISAMIDE | 5.0000 | 7.77 | 0.000000242 | 0.000009402 | 0.6 | 49.82 |
| TRIDECEN 2 NITRILE | 0.4000 | 6.43 | 0.000000137 | 0.000000353 | 6.7 | 0.36 |
| | | | | | % Citrus | 95.02 |
| | | | | | % Fruity | 0.00 |

The perfume “Sustained Release Citrus-Fruity” provides a linear sustained citrus note during rinse-off along with a less dominant linear fruity nuance as well in various applications such as body-wash, conditioners etc.

### C. Delayed Citrus-Fruity Linear Release Perfume

The perfume “Delayed Citrus-Fruity Linear Release” is intended to maximize deposition of fragrance whilst providing the consumer with an impactful release of a citrus fragrance along with a less dominant, secondary linear fruity note during rinse-off.

It is engineered based on odorants' physico kinetic properties as described in the preceding examples for Delayed Citrus-Green Perfume. The analysis of Delayed Citrus-Fruity Linear Release Perfume is shown below in table 23:

TABLE 23

| | Parts | γ | φ | Ω | ODT | % Odor |
|---|---|---|---|---|---|---|
| Water Release I | | | | | | |
| d-LIMONINE | 2.0000 | 8200.76 | 0.00157182 | 25.78023899 | 430 | 100.00 |
| | | | | | % Citrus | 100.00 |
| | | | | | % Fruity | 0.00 |
| Water Release II | | | | | | |
| MANZANATE | 0.5000 | 5288.42 | 0.00111633 | 2.95182335 | 50 | 1.13 |
| ETHYL 2-METHYLBUTYRATE | 0.0500 | 12827.56 | 0.00436154 | 2.79739384 | 20 | 0.28 |
| CITRONELLAL | 10.0000 | 1345.09 | 0.00012444 | 1.67381346 | 33 | 34.14 |
| CITRONELLYL NITRILE | 7.5000 | 913.04 | 0.00005329 | 0.29193475 | 71 | 11.90 |
| ORTHOLATE | 8.0000 | 564.56 | 0.00006262 | 0.28282987 | 58 | 15.54 |
| ALLYL CAPROATE | 0.5000 | 1736.67 | 0.00021070 | 0.15302701 | 4.6 | 12.25 |
| CITRAL | 2.5000 | 857.09 | 0.00006235 | 0.13361561 | 12 | 23.47 |
| RHUBAFURAN | 5.0000 | 476.06 | 0.00003758 | 0.05945177 | 440 | 1.28 |
| | | | | | % Citrus | 70.80 |
| | | | | | % Fruity | 29.20 |
| Water Release III | | | | | | |
| APPLINAL | 1.5000 | 554.79 | 0.00008246 | 0.05197587 | 55 | 1.02 |
| APHERMATE | 1.5000 | 589.82 | 0.00005808 | 0.04959973 | 100 | 0.58 |
| ALDEHYDE C10 | 1.0000 | 818.28 | 0.00008057 | 0.04966423 | 8.7 | 5.58 |
| LINALYL ACETATE | 1.1000 | 617.72 | 0.00006451 | 0.04383354 | 450 | 0.09 |
| ALLYL HEPTANOATE | 0.5000 | 711.58 | 0.00008076 | 0.02161678 | 58 | 0.32 |
| MEFRANAL | 8.5000 | 84.76 | 0.00000349 | 0.01300043 | 3.5 | 88.27 |
| DIMETHYL BENZYL CARBINYL ACETATE | 2.0000 | 249.93 | 0.00001784 | 0.00891854 | 18 | 4.15 |
| | | | | | % Citrus | 93.94 |
| | | | | | % Fruity | 6.06 |
| Water Release IV | | | | | | |
| OXANE | 0.5000 | 294.10 | 0.00003802 | 0.00559049 | 58 | 0.04 |
| GARBAMIDE | 12.0000 | 27.84 | 0.00000140 | 0.00058309 | 0.5 | 99.79 |
| PHENOXY ETHYL ISOBUTYRATE | 4.0000 | 52.67 | 0.00000254 | 0.00053465 | 120 | 0.17 |
| | | | | | % Citrus | 99.83 |
| | | | | | % Fruity | 0.17 |
| Water Release IV | | | | | | |
| OXANE | 0.5000 | 294.10 | 0.00003802 | 0.00559049 | 58 | 0.04 |
| GARBAMIDE | 12.0000 | 27.84 | 0.00000140 | 0.00058309 | 0.5 | 99.79 |
| PHENOXY ETHYL ISOBUTYRATE | 4.0000 | 52.67 | 0.00000254 | 0.00053465 | 120 | 0.17 |
| | | | | | % Citrus | 99.83 |
| | | | | | % Fruity | 0.17 |
| Water Release V | | | | | | |
| CITRATHAL | 5.5000 | 39.32 | 0.00000192 | 0.00042206 | 5.3 | 4.03 |
| DIMETHYL BENZYL CARBINYL BUTYRATE | 3.0000 | 39.87 | 0.00000185 | 0.00022126 | 42 | 0.28 |
| GAMMA UNDECALACTONE | 5.0000 | 42.98 | 0.00000151 | 0.00009754 | 0.7 | 27.74 |
| NECTARYL | 7.0000 | 13.54 | 0.00000051 | 0.00004885 | 0.4 | 87.98 |
| | | | | | % Citrus | 71.99 |
| | | | | | % Fruity | 28.01 |
| Water Release VI | | | | | | |
| PARADISAMIDE | 8.5000 | 7.77 | 0.00000024 | 0.00001880 | 0.6 | 48.36 |
| TRIDECEN 2 NITRILE | 0.8500 | 8.43 | 0.00000014 | 0.00000075 | 6.7 | 0.43 |
| ETHYL METHYL PHENYL GLYCIDATE | 1.5000 | 28.35 | 0.000000116 | 0.00001533 | 0.1 | 51.21 |
| | | | | | % Citrus | 48.79 |
| | 100.0000 | | | | % Fruity | 51.21 |

The above perfume Delayed Citrus-Fruity Linear Release provides the consumer with a perceived impactful citrus linear release during rinse-off along with a secondary linear fruity nuance whilst resulting in maximum deposition of fragrance as well.

### Citrus-Floral Perfumes

The following examples are for Citrus-Fragrance family of perfumes which result in a linear impactful release of a citrus note along with a secondary floral fragrance in the presence of large water quantities.

Following the rationale provided in earlier examples, perfumes were engineered for flash release, sustained release and delayed release according to their intended application and usage.

TABLE 24
Flash Release Citrus-Floral

| | | | | | | % Odor |
|---|---|---|---|---|---|---|
| | Parts | γ | φ | Ω | odt | Contribution |
| Water Release Group 1 | | | | | | |
| d-LIMONINE | 42.25 | 8200.76 | 0.00157182 | 544.60754865 | 430 | 100.00 |
| | | | | | % Citrus | 100.00 |
| | | | | | % Floral | 0.00 |
| Water Release Group 2 | | | | | | |
| LINALOOL | 12.00 | 844.41 | 0.00004775 | 0.36924192 | 20 | 63.75 |
| DIHYDRO MYRCENOL | 5.00 | 866.55 | 0.00007332 | 0.31765764 | 810 | 0.66 |
| CITRONELLAL | 1.50 | 1345.09 | 0.00012444 | 0.25107202 | 33 | 4.83 |
| CITRONELLYL NITRILE | 4.50 | 913.04 | 0.00004329 | 0.21895107 | 71 | 8.73 |
| CIS 3 HEXENYL ACETATE | 0.50 | 1384.27 | 0.00013021 | 0.09012156 | 170 | 0.31 |
| ALDEHYDE C 8 (OCTANAL) | 0.05 | 3630.08 | 0.00045510 | 0.07434161 | 4.6 | 1.04 |
| ALDEHYDE C10 (DECANAL) | 1.43 | 818.26 | 0.00006057 | 0.07087684 | 6.7 | 22.68 |
| | | | | | % Citrus | 35.94 |
| | | | | | % Floral | 63.75 |
| Water Release Group 3 | | | | | | |
| RHUBAFURAN | 3.00 | 476.06 | 0.00003758 | 0.05367106 | 440 | 0.16 |
| ETHYL LINALOOL | 5.70 | 275.63 | 0.00001520 | 0.02387786 | 120 | 1.12 |
| ROSSITOL | 3.00 | 303.80 | 0.00002075 | 0.01891460 | 440 | 0.16 |
| IONONE-BETA | 2.50 | 311.32 | 0.00002357 | 0.01834091 | 0.6 | 98.31 |
| PHENYL ETHYL ACETATE | 1.50 | 384.06 | 0.00002819 | 0.01624022 | 150 | 0.24 |
| CIS 3 HEXENOL | 0.05 | 1569.11 | 0.00016354 | 0.01283060 | 90 | 0.01 |
| | | | | | % Citrus | 1.44 |
| | | | | | % Floral | 98.31 |
| Water Release Group 4 | | | | | | |
| LILIAL | 7.50 | 104.63 | 0.00000569 | 0.00446800 | 0.93 | 100.00 |
| | | | | | % Citrus | 0.00 |
| | | | | | % Floral | 100.00 |
| Water Release Group 5 | | | | | | |
| GARDAMIDE | 1.50 | 88.47 | 0.00000316 | 0.00031550 | 24 | 16.62 |
| MEFRANAL | 0.52 | 84.76 | 0.00000349 | 0.00015364 | 17 | 8.13 |
| CITRATHAL | 1.50 | 39.32 | 0.00000192 | 0.00011511 | 5.3 | 75.25 |
| | | | | | % Citrus | 100.00 |
| | | | | | % Floral | 0.00 |
| Water Release Group 6 | | | | | | |
| PARADISAMIDE | 2.50 | 7.77 | 0.00000024 | 0.00000470 | 0.6 | 38.40 |
| METHYL DIHYDRO JASMONA | 1.50 | 8.40 | 0.00000029 | 0.00000360 | 0.23 | 60.10 |
| TRIDECEN 2 NITRILE | 0.50 | 6.43 | 0.00000014 | 0.00000044 | 8.7 | 0.69 |
| CALYXOL | 1.50 | 1.23 | 0.00000004 | 0.00000008 | 17 | 0.81 |
| | | | | | % Citrus | 39.09 |
| | | | | | % Floral | 60.918 |
| Perfume Total | 100.00 | | | | | |

TABLE 25
Sustained Citrus-Floral Linear Release

| | | | | | | % Odor |
|---|---|---|---|---|---|---|
| | Parts | γ | φ | Ω | odt | Contribution |
| Water Release Group 1 | | | | | | |
| d-LIMONINE | 10.00 | 8200.76 | 0.001571820 | 128.901194947 | 430 | 100.00 |
| | | | | | % Citrus | 100.00 |
| | | | | | % Floral | 0.00 |
| Water Release Group 2 | | | | | | |
| CITRONELLOL | 12.00 | 888.56 | 0.000088703 | 0.895227041 | 29 | 53.41 |
| DIHYDRO MYRCENOL | 5.00 | 866.55 | 0.000073318 | 0.317657635 | 810 | 0.80 |
| CITRONELLAL | 1.50 | 1345.09 | 0.000124439 | 0.251072020 | 33 | 5.87 |
| CITRONELLYL NITRILE | 4.50 | 913.04 | 0.000053290 | 0.218951073 | 71 | 8.18 |
| BENZYL ACETATE | 5.00 | 884.29 | 0.000059238 | 0.196748700 | 252 | 2.58 |
| CIS 3 HEXENYL ACETATE | 0.50 | 1384.27 | 0.000130208 | 0.090121564 | 170 | 0.38 |
| ALDEHYDE C 8 (OCTANAL) | 0.05 | 3630.08 | 0.000455096 | 0.074341612 | 4.6 | 1.28 |
| ALDEHYDE C10 (DECANAL) | 1.43 | 818.26 | 0.000080573 | 0.070876843 | 6.7 | 27.55 |
| | | | | | % Citrus | 43.65 |
| | | | | | % Floral | 55.97 |
| Water Release Group 3 | | | | | | |
| RHUBAFURAN | 3.00 | 476.06 | 0.000037580 | 0.053871059 | 440 | 0.16 |
| GERANYL ACETATE | 5.00 | 314.92 | 0.000022992 | 0.038203472 | 89 | 1.88 |
| ETHYL LINALOOL | 5.00 | 275.63 | 0.000015198 | 0.020945492 | 120 | 0.97 |
| ROSSITOL | 3.00 | 303.30 | 0.000020753 | 0.018914596 | 440 | 0.18 |
| IONONE-BETA | 2.50 | 311.32 | 0.000023566 | 0.018340905 | 0.6 | 96.79 |
| PHENYL ETHYL ACETATE | 1.50 | 384.06 | 0.000028191 | 0.018240221 | 150 | 0.23 |
| CIS 3 HEXENOL | 0.05 | 1569.11 | 0.000183540 | 0.012830600 | 90 | 0.01 |
| | | | | | % Citrus | 1.28 |
| | | | | | % Floral | 98.70 |
| Water Release Group 4 | | | | | | |
| LILIAL | 5.00 | 104.63 | 0.000005694 | 0.002978669 | 0.93 | 99.39 |
| HYDROXYCITRONELLAL | 8.95 | 45.82 | 0.000001620 | 0.000664438 | 270 | 0.61 |
| | | | | | % Citrus | 0.61 |
| | | | | | % Floral | 99.39 |
| Water Release Group 5 | | | | | | |
| GARDAMIDE | 1.50 | 66.47 | 0.000003184 | 0.000315504 | 24 | 16.62 |
| MEFRANAL | 0.52 | 84.76 | 0.000003486 | 0.000153837 | 17 | 8.13 |
| CITRATHAL | 1.50 | 39.32 | 0.000001915 | 0.000115109 | 5.3 | 75.25 |
| | | | | | % Citrus | 100.00 |
| | | | | | % Floral | 0.00 |
| Water Release Group 6 | | | | | | |
| METHYL DIHYDRO JASMONATE | 10.00 | 8.40 | 0.000000286 | 0.000024021 | 0.23 | 74.03 |
| PARADISAMIDE | 9.00 | 7.77 | 0.000000242 | 0.000016924 | 0.6 | 25.54 |
| TRIDECEN 2 NITRILE | 0.50 | 6.43 | 0.000000137 | 0.000000441 | 6.7 | 0.13 |
| CALYXOL | 3.00 | 1.23 | 0.000000041 | 0.000000151 | 17 | 0.30 |
| Perfume Total | 100.00 | | | | % Citrus | 25.67 |
| | | | | | % Floral | 74.33 |

TABLE 27
Delayed Citrus-Floral Linear Release

| | | | | | | % Odor |
|---|---|---|---|---|---|---|
| | Parts | γ | φ | Ω | odt | Contribution |
| Water Release Group 1 | | | | | | |
| d-LIMONINE | 2.50 | 8200.76 | 0.001571820 | 32.22529874 | 430 | 100.00 |
| | | | | | % Citrus | 100.00 |
| | | | | | % Floral | 0.00 |
| Water Release Group 2 | | | | | | |
| CITRONELLOL | 5.50 | 868.56 | 0.000066703 | 0.31884573 | 29 | 70.80 |
| DIHYDRO MYRCENOL | 2.50 | 866.55 | 0.000073316 | 0.15882882 | 810 | 1.15 |
| CITRONELLAL | 1.50 | 1345.09 | 0.000124439 | 0.25107202 | 33 | 18.97 |
| CITRONELLYL NITRILE | 0.50 | 913.04 | 0.000053290 | 0.02432790 | 71 | 2.83 |
| BENZYL ACETATE | 2.50 | 664.29 | 0.000059238 | 0.09837435 | 252 | 3.70 |
| CIS 3 HEXENYL ACETATE | 0.50 | 1384.27 | 0.000130208 | 0.09012158 | 170 | 1.10 |
| ALDEHYDE C 8 (OCTANAL) | 0.05 | 3630.08 | 0.000455096 | 0.07434161 | 4.6 | 3.85 |
| | | | | | % Citrus | 74.50 |
| | | | | | % Floral | 24.40 |
| Water Release Group 3 | | | | | | |
| ALDEHYDE C10 (DECANAL) | 0.50 | 818.26 | 0.000060573 | 0.02478211 | 8.7 | 1.70 |
| RHUBAFURAN | 3.00 | 475.05 | 0.000037580 | 0.05367106 | 440 | 0.18 |
| GERANYL ACETATE | 5.00 | 314.92 | 0.000022992 | 0.03620347 | 69 | 1.65 |
| ETHYL LINALOOL | 5.00 | 275.63 | 0.000015198 | 0.02094549 | 120 | 0.95 |
| ROSSITOL | 3.00 | 303.80 | 0.000020753 | 0.01891460 | 440 | 0.18 |
| IONONE-BETA | 2.50 | 311.32 | 0.000023566 | 0.01834091 | 0.6 | 95.14 |
| PHENYL ETHYL ACETATE | 1.50 | 384.05 | 0.000028191 | 0.01624022 | 150 | 0.23 |
| CIS 3 HEXENOL | 0.05 | 1569.11 | 0.000163540 | 0.01283060 | 90 | 0.01 |
| | | | | | % Citrus | 2.97 |
| | | | | | % Floral | 97.02 |
| Water Release Group 4 | | | | | | |
| LILIAL | 5.00 | 104.63 | 0.000005894 | 0.00297867 | 0.93 | 90.12 |
| HYDROXYCITRONELLAL | 10.00 | 45.82 | 0.000001620 | 0.00074239 | 270 | 0.62 |
| GARDAMIDE | 5.50 | 55.47 | 0.000003164 | 0.00115685 | 24 | 3.84 |
| MEFRANAL | 5.50 | 84.76 | 0.000003485 | 0.00182501 | 17 | 5.42 |
| | | | | | % Citrus | 9.88 |
| | | | | | % Floral | 90.12 |
| Water Release Group 5 | | | | | | |
| CITRATHAL | 5.00 | 39.32 | 0.000001915 | 0.00038370 | 5.3 | 1.43 |
| METHYL DIHYDRO JASMONATE | 15.00 | 8.40 | 0.000000286 | 0.00003603 | 0.23 | 98.57 |
| | | | | | % Citrus | 1.43 |
| | | | | | % Floral | 98.57 |
| Water Release Group 6 | | | | | | |
| PARADISAMIDE | 4.00 | 7.77 | 0.000000242 | 0.00000752 | 0.5 | 88.08 |
| TRIDECEN 2 NITRILE | 3.50 | 6.43 | 0.000000137 | 0.00000309 | 6.7 | 6.75 |
| BENZYL SALICYLATE | 5.00 | 1.73 | 0.000000070 | 0.00000059 | 21 | 3.07 |
| CALYXOL | 5.40 | 1.23 | 0.000000041 | 0.00000027 | 17 | 4.10 |
| | | | | | % Citrus | 92.82 |
| | | | | | % Floral | 7.18 |

1.-12. (canceled)

13. A method of formulating a citrus perfume composition for use in rinse-off systems such that said composition provides a continuous citrus note during rinse-off or upon water dilution, said composition comprising odorants in at least three different water release groups, each water release group comprising at least 30% citrus descriptor for its overall odour, said method comprising calculating values of odour threshold detection, acceleration and water release values for a group of odorants, to provide water release groups including a flash water release group having an acceleration value greater than 900, a sustained water release group having an acceleration value of 100 to 900; and a delayed water release group having an acceleration value of less than 100 and selecting odorants from said water release groups to provide a citrus perfume composition
(a) containing at least 20 wt % of the flash release group and not more than 30 wt % of delayed water release group;
(b) at least 30 wt % of sustained water release group; or
(c) at least 30 wt % of delayed water release group.

14. The method of claim 13 wherein said composition comprises odorants in the different water release groups such that each water release group comprises at least 20% green descriptor for its overall odour.

15. The method of claim 13 wherein said composition comprises odorants in the different water release groups such that each water release group comprises at least 20% fruity descriptor for its overall odour.

16. The method of claim 13 wherein said composition comprises odorants in the different water release groups such that each water release group comprises at least 20% floral descriptor for its overall odour.

17. The method of claim 13 wherein the odorants are selected so that said composition includes 30% odorant with an acceleration value of greater than 900, and no more than 15 wt % of odorants with an acceleration value of less than 100.

18. The method of claim 13 wherein the composition comprises at least 40 wt % of odorants with an acceleration value of from 100 to 900.

19. The method of claim 13 wherein the composition comprises at least 40 wt % of odorants with an acceleration value of less than 100.

20. The method of claim 13 wherein said composition is included in a rinse-off consumer composition.

21. The method of claim 13 wherein the composition is added to a surface cleaner, detergent, shampoo or conditioner.

22. A method of formulating a perfume composition for wash-off systems comprising determining an odor detection threshold, an acceleration term and water release values for a group of odorants and selecting from these values the appropriate combination of odorants to provide a continuous citrus note upon water dilution, optionally with a linear release of a secondary, less prominent note of fruity, green or floral odor.
